(19) United States
(12) Patent Application Publication
(10) Pub. No.: US 2023/0183884 A1
(43) Pub. Date: Jun. 15, 2023

(54) A METHOD FOR CRISPR LIBRARY SCREENING

(71) Applicant: CHINA AGRICULTURAL UNIVERSITY, Beijing (CN)

(72) Inventors: Sen WU, Beijing (CN); Chunlong XU, Beijing (CN); Xiaolan QI, Beijing (CN); Xuguang DU, Beijing (CN); Huiying ZOU, Beijing (CN)

(21) Appl. No.: 16/464,660

(22) PCT Filed: Nov. 30, 2016
(86) PCT No.: PCT/CN2016/107952

§ 371 Date: May 28, 2019

(51) Int. Cl. C40B 40/08; C12N 15/85; C12N 15/11; C12N 9/22

(52) U.S. Cl. CPC C40B 40/08; C12N 15/85; C12N 15/11; C12N 9/22; C12N 2800/90; C12N 2310/20

(57) ABSTRACT

CRISPR/Cas9 is becoming an increasingly important tool to functionally annotate genomes. However, since genome-wide CRISPR/Cas9 libraries are mostly constructed in lentiviral vectors, in vivo applications are severely limited due to difficulties in delivery. Here we examined the piggyBac (PB) transposon as an alternative vehicle to deliver a guide RNA (gRNA) library for in vivo screening. Although tumor induction has previously been achieved in mice by targeting cancer genes with the CRISPR/Cas9 system, in vivo genome-scale screening has not been reported. With our PB-CRISPR libraries, we conducted an in vivo genome-wide screen in mice and identified genes mediating liver tumorigenesis, including known and novel tumor suppressor genes (TSGs), Our results demonstrate that PB can be a simple and non-viral choice for efficient in vivo delivery of CRISPR libraries.

This application is a U.S. National Stage entry of PCT Application No. PCT/CN2016/107952, filed Nov. 30, 2016, the contents of which are incorporated herein by reference.

### TECHNICAL FIELD

The present invention relates to the technology of vector construction, genome-wide screens for mutagenesis and especially relates to the piggyBac (PB) transposon as a vehicle to deliver a guide RNA library and designed for in vivo screens.

### TECHNICAL BACKGROUND

For the past decade, transposon mutagenesis and RNA interference mediated screens have been the main methods for in vivo screening and validation of cancer genes in mice (Bard-Chapeau E A, et al. Nature genetics 46(1):24-32.(2014); Carlson C M, et al. Proceedings of the National Academy of Sciences of the United States of America 102(47): 17059-17064. (2005); Keng V W, et al. Nature biotechnology 27(3):264-274.(2009); Dupuy A J, et al. Nature 436(7048):221-226.(2005); Zender L, et al. Cell 135(5):852-864.(2008); Schramek D, et al. Science 343(6168):309-313.(2014)). However, due to their low efficiency, these two methods have not been widely used. Recently, CRISPR/Cas9 has been developed as an efficient mutagenesis tool (Cong L, et al. Science 339(6121):819-823.(2013); Mali P, et al. Science 339(6121):823-826.(2013)) and was quickly adapted for as a technique for in vivo tumor induction and validation of cancer genes (Sanchez-Rivera F J, et al. Nature 516(7531):428-+.(2014); Chiou S H, et al. Genes & Development 29(14):1576-1585.(2015); Zuckermann M, et al. Nature Communications 6:9.(2015); Maddalo D, et al. Nature 516(7531):423-+. (2014); Xue W, et al. Nature 514(7522):380-384.(2014); Weber J, et al. Proceedings of the National Academy of Sciences of the United States of America 112(45):13982-13987.(2015)). By transplanting CRISPR library transduced cancer cells into immuno-compromised mice, several genes involved in growth and metastasis of human lung cancer were identified (Chen S D, et al. Cell 160(6):1246-1260.(2015)). However, direct in vivo genome-wide CRISPR screening has not been successfully achieved due to limitations of current lentiviral delivery methods (Chen S D, et al. Cell 160(6):1246-1260.(2015); Sanchez-Rivera F J, et al. Nature 516(7531):428-+.(2014)). Furthermore, all previous screening strategies suffer from several drawbacks. These screens typically start with an immuno-comprised genetic background or a genetic background carrying multiple pre-engineered mutations, and thus the results may not be applicable to wild-type mice (Bard-Chapeau E A, et al. Nature genetics 46(1):24-32. (2014); Zender L, et al. Cell 135(5):852-864.(2008)). They usually need >1 year to obtain tumors (Weber J, et al. Proceedings of the National Academy of Sciences of the United States of America 112(45):13982-13987.(2015); Bard-Chapeau E A, et al. Nature genetics 46(1):24-32. (2014); Keng V W, et al. Nature biotechnology 27(3):264-274.(2009)).

In summary, the key for achieving direct in vivo genome-wide CRISPR library screening and/or better in vitro screening is the high efficiency of a delivery system. However, all previously tested systems have not been able to achieve direct in vivo genome-wide CRISPR library screening. Therefore, there is a strong need for an alternative delivery system that can overcome these shortcomings and can be used for direct in vivo CRISPR library screening, as well as more efficient in vitro screening.

### SUMMARY OF INVENTION

The present invention relates to the technology of vector construction, genome-wide screens for mutagenesis and especially relates to the piggyBac (PB) transposon as an alternative vehicle to deliver a guide RNA library and designed for in vivo screens. The present invention provides a method of in vivo genome-scale screening for tumorigenesis.

In one aspect, the present invention provides a genome wide library comprising:

a plurality of PB-mediated CRISPR system polynucleotide, comprising minimal guide RNAs flanked by minimal piggyBac inverted repeat elements, and said guide sequences are capable of targeting a plurality of target sequences of interest in a plurality of genomic loci in a population of eukaryotic cells, tissues, or organisms.

The aforesaid library, wherein the population of eukaryotic cells is a population of mammalian cells such as mouse cells or human cells.

The aforesaid library, wherein the population of eukaryotic cells is a population of any kind of cells such as fibroblast.

The aforesaid library, wherein the population of tissues is a population of any kind of the non-reproductive tissues such as liver or lungs.

The aforesaid library, wherein the population of organisms is a population of mouse.

The aforesaid library, wherein the target sequence in the genomic locus is a coding sequence.

The aforesaid library, wherein gene function of said target sequence is altered by said targeting.

The aforesaid library, wherein said targeting results in a knockout of gene function.

The aforesaid library, wherein the targeting is of the entire genome.

In some embodiment, wherein the knockout of gene function is achieved in a plurality of unique genes which function in mediating tumorigenesis, anti-aging, and longevity.

In a specific embodiment, wherein said unique gene is tumor suppressor gene.

The invention also provides a method of in vivo genome-scale screening comprising:

(a) introducing into a mammal containing and expressing a RNA polynucleotide having a target sequence,

(b) encoding at least one gene product of a PB-mediated CRISPR system comprising one or more vectors comprising:

(i) a first polynucleotide encoding a Cas9 protein, or a variant thereof or a fusion protein therewith,

(ii) a second polynucleotide encoding a PB transposase, or a variant thereof or a fusion protein therewith,

(iii) a third polynucleotide library of claims 1-11,

wherein components (i), (ii), and (iii) are located on same or different vectors of the system,

whereby PB transposase introduce guide RNA into genomes, the guide RNA targets the target sequence an Cas9 protein generates at least one site specific break is repaired through a cellular repair mechanism,

(c) amplifying and sequencing the genomic DNA from said mammal.

The aforesaid method, wherein gene function of said gene product is altered by said system.

The aforesaid method, wherein said system results in a knockout of gene function.

The aforesaid method, wherein the knockout of gene function is achieved in a plurality of unique genes which function in mediating tumorigenesis, anti-aging, and longevity.

The aforesaid method, wherein said mammal in step (a) expresses at least one oncogene or knockouts at least one tumor suppresser gene to generate a sensitized background for screening without tumor formation.

The aforesaid method, wherein said oncogene is NRAS with dominant G12V mutation.

The aforesaid method, wherein said tumor suppresser gene is selected from the group consists of Cdkn2b, Trp53, Klf6, miR-99b, Clec5a, SelIl2, Lgals7, Pml, Ptgdr, Tspan32, Fat4, Pik3ca, Pdlim4, Cxcl12, Lrig1, Batf2, Prodh2, Chst10, Dims1, Ephb4, Timp3, Hrasls, Banp, and Cyb561d2.

In some embodiment, wherein said mammal is mouse.

In a specific embodiment, wherein PB-mediated CRISPR system is introduced into mouse by hydrodynamic tail vein injection.

In a specific embodiment, wherein PB-mediated CRISPR system is introduced by transfection in vivo such as nanoparticles and electroporation.

Significance

Since genome-wide CRISPR/Cas9 libraries are mostly constructed in lentiviral vectors, direct in vivo screening have not been possible due to low efficiency in delivery. Here we examined the piggyBac (PB) transposon as an alternative vehicle to deliver a guide RNA (gRNA) library for in vivo screening. Through hydrodynamic tail vein injections, we delivered a PB-CRISPR library into mouse liver. Rapid tumor formation could be observed in less than 2 months. By sequencing analysis of PB mediated gRNA insertions, we identified corresponding genes mediating tumorigenesis. Our results demonstrate that PB is a simple and non-viral choice for efficient in vivo delivery of CRISPR libraries for phenotype-driven screens.

### BRIEF DESCRIPTION OF DRAWINGS

FIG. 1. PB-CRISPR vectors and validation by targeting Tet1 and Tet2 in mouse iPS cells. (A) PB based CRISPR vectors. pCRISPR-sg4, sgRNA expressing vector with neo gene; pCRISPR-sg5, sgRNA expressing vector with puromycin gene. (B) pCRISPR-S10, PB plasmid expressing Dox inducible Cas9; pCRISPR-sg6-Tet1/Tet2, pCRISPR-sg6 based plasmids expressing Tet1 or Tet2 sgRNA. (C) PCR-RFLP analysis of Tet1/Tet2 loci targeted by pCRISPR-sg6-Tet1/Tet2. Expected mutations would eliminate the SacI or EcoRV site in Tet1 and Tet2, respectively. The target regions (˜500 bp) of Tet1 or Tet2 were amplified by PCR. PCR products were digested with corresponding enzymes. Results showed the successful targeting in Tet1-clone 1, Tet1-clone 2 and Tet2-clone 2.(D) Sequencing results of Tet1/Tet2 sgRNA targeted loci. Sequencing results for Tet1-clone 1 revealed a 4 bp deletion in one allele and a 1 bp deletion in another, resulting in elimination of the SacI site. Sequencing results for Tet1-clone 2 revealed mutations in both alleles, with a 3 bp deletion in one and 1 bp insertion in another resulting in elimination of the SacI site. Sequencing results for Tet2-clone 2, with an 8 bp deletion in one allele and a 14 bp deletion in another, resulting in elimination of the EcoRV site.

FIG. 2. PB-CRISPR library construction and in vivo delivery. (A) Work flow of PB-CRISPR library construction. PB, piggyBac transposon; PB 3′TR/5′TR, 3′ and 5′ terminal repeat sequence of PB; U6, human U6 promoter; ccdB, a toxin gene for bacteria; p(T), poly T terminator sequence; sgRNA scaffold, scaffold sequence for chimeric sgRNA; 20 nt guide, guide sequence for chimeric sgRNA. (B) PB-CRISPR-M2 library correlated well (r²=0.83) with the GeCKOv2 mouse library in terms of total gRNA distribution, and 95% of sgRNAs in GeCKOv2 can be found in PB-CRISPR-M2. (C) In vivo delivery of PB-CRISPR-M2 library by tail vein injection. pPB-IRES-EGFP, PB plasmid expressing IRES-EGFP. pCAG-PBase expresses CAG promoter driven PBase. Mice were injected with PB-CRISPR-M2 library, pPB-IRES-EGFP, and pCAG-PBase. Control group was injected without pCAG-PBase. Liver samples were evaluated for GFP expression and used for NGS at 14 days post injection. Scale bars, 2 mm.

FIG. 3. Transfection of mouse testis with PB vectors. (A) In vivo transfection of testis with PB vectors by electroporation. Control testis was injected with Trypan blue only. Experiment testis was injected with pPB-IRES-EGFP, and pCAG-PBase. (B) Twenty-four hours after electroporation, testes were examined for GFP expression. Dashed line indicates testis without transfection by PB vectors. Scale bar, 1 mm.

FIG. 4. Quantitative RT-PCR for transgene expression in mouse liver injected with PB vectors. (A) Schematic maps of PB vectors used in the screening experiment. Mice (n=3) were injected with pPB-hNRAS^{G12V}, pCRISPR-W9-Cdkn2a-sgRNA and pCAG-PBase. Control mice (n=3) were injected with saline only. (B) Cas9 expression in mouse liver samples. (C) hNRAS^{G12V} expression in mouse liver samples.

FIG. 5. Successful induction of liver tumors in mice using PB-CRISPR library screening. (A) Procedure to conduct a PB-CRISPR screen for genes promoting tumorigenesis in liver. Liver delivery of PB-CRISPR system was carried out with hydrodynamic tail vein injection. (B) Representative liver tumors obtained from the screen. Scale bar, 2 mm (C) Histology and immunohistochemistry analysis of a moderately differentiated intrahepatic cholangiocarcinoma (ICC). H&E slides show that tumor cells have a tubular growth pattern, in contrast to the normal liver tissue. Tumor cells express CK19 and Ki67. Scale bars, 100 μm for low magnification, 50 μm for high magnification.

FIG. 6. Histology and IHC analysis of representative tumors. (A) A moderately differentiated intrahepatic cholangiocarcinoma (ICC). Tumor cells express cytokeratin markers AE1/AE3. The surrounding stroma can be identified by SMA, Vimentin and Collagen-4 (Coll4) staining (B) A representative undifferentiated pleomorphic sarcoma (UPS). Tumor cells were negative for AFP and CK19, but have high proliferative capacity, as shown by Ki67 staining Scale bars, 100 μm for low magnification, 50 μm for high magnification.

FIG. 7. Summary of sgRNA content of 18 tumors. PCR was performed on each tumor for NGS. On average 15 library sgRNAs were present in each tumor. Among the total of 271 sgRNAs isolated in 18 tumors, 26 sgRNA targeting known TSGs were indicated for the corresponding tumor (two-sided Fisher's exact test, P<0.01). Cdkn2b and Trp53, were targeted 4 and 2 times, respectively.

FIG. 8. Validation of sgRNAs for Trp53 and Cdkn2b. (A) Validation of Trp53 and Cdkn2b sgRNAs for liver tumorigenesis in mice. Typical tumors are shown for each group. Histology and immunohistochemistry analyses indicated they were intrahepatic cholangiocarcinomas. In the Trp53 group with Cdkn2a-sgRNA, when mice were examined at day 21 post injection, 10 out of 11 mice had tumors in the liver (P<0.01, χ² test). In the Trp53 group without Cdkn2a-sgRNA, 8 out of 11 mice had liver tumors at 28 days (P<0.01, χ² test). In the Cdkn2b group, 4 out of 11 mice developed liver tumors (P<0.01, χ² test) at 45 days post injection. Scale bars, 2 mm for tumors, 100 μm for H&E, 50 μm for CK19. (B) Representative Sanger sequencing results of target regions of Trp53 (frameshift indels), Cdkn2b (frameshift inde1 and nonsense mutation T) in the tumors.

### DETAILED DESCRIPTION

The present invention will be further illustrated below with reference to the specific examples. It should be understood that these examples are only used to describe the invention but not to limit the scope of the invention. The experimental methods with no specific conditions described in the following examples are generally performed under conventional conditions, and the materials used without specific description are purchased from common chemical reagents corporation.

Before describing the invention in detail, it is to be understood that this invention is not limited to particular biological systems or cell types. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes combinations of two or more cells, or entire cultures of cells; reference to “a polynucleotide” includes, as a practical matter, many copies of that polynucleotide. Unless defined herein and below in the reminder of the specification, all technical and scientific terms used herein have the same meaning as commonly understood by one of ordinary skill in the art to which the invention pertains.

As used herein, the terms “polynucleotide”, “nucleic acid,” “oligonucleotide”, “oligomer”, “oligo” or equivalent terms, refer to molecules that comprises a polymeric arrangement of nucleotide base monomers, where the sequence of monomers defines the polynucleotide. Polynucleotides can include polymers of deoxyribonucleotides to produce deoxyribonucleic acid (DNA), and polymers of ribonucleotides to produce ribonucleic acid (RNA). A polynucleotide can be single- or double-stranded. When single stranded, the polynucleotide can correspond to the sense or antisense strand of a gene. A single-stranded polynucleotide can hybridize with a complementary portion of a target polynucleotide to form a duplex, which can be a homoduplex or a heteroduplex.

The length of a polynucleotide is not limited in any respect. Linkages between nucleotides can be internucleotide-type phosphodiester linkages, or any other type of linkage. A polynucleotide can be produced by biological means (e.g., enzymatically), either in vivo (in a cell) or in vitro (in a cell-free system). A polynucleotide can be chemically synthesized using enzyme-free systems. A polynucleotide can be enzymatically extendable or enzymatically non-extendable.

By convention, polynucleotides that are formed by 3′-5′ phosphodiester linkages (including naturally occurring polynucleotides) are said to have 5′-ends and 3′-ends because the nucleotide monomers that are incorporated into the polymer are joined in such a manner that the 5′ phosphate of one mononucleotide pentose ring is attached to the 3′ oxygen (hydroxyl) of its neighbor in one direction via the phosphodiester linkage. Thus, the 5′-end of a polynucleotide molecule generally has a free phosphate group at the 5′ position of the pentose ring of the nucleotide, while the 3′ end of the polynucleotide molecule has a free hydroxyl group at the 3′ position of the pentose ring. Within a polynucleotide molecule, a position that is oriented 5′ relative to another position is said to be located “upstream”, while a position that is 3′ to another position is said to be “downstream”. This terminology reflects the fact that polymerases proceed and extend a polynucleotide chain in a 5′ to 3′ fashion along the template strand. Unless denoted otherwise, whenever a polynucleotide sequence is represented, it will be understood that the nucleotides are in 5′ to 3′ orientation from left to right.

As used herein, it is not intended that the term “polynucleotide” be limited to naturally occurring polynucleotide structures, naturally occurring nucleotides sequences, naturally occurring backbones or naturally occurring internucleotide linkages. One familiar with the art knows well the wide variety of polynucleotide analogues, unnatural nucleotides, non-natural phosphodiester bond linkages and internucleotide analogs that find use with the invention.

As used herein, the term “gene” generally refers to a combination of polynucleotide elements, that when operatively linked in either a native or recombinant manner, provide some product or function. The term “gene” is to be interpreted broadly, and can encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” encompasses mRNA, cDNA and genomic forms of a gene.

In some aspects, the genomic form or genomic clone of a gene includes the sequences of the transcribed mRNA, as well as other non-transcribed sequences which lie outside of the transcript. The regulatory regions which lie outside the mRNA transcription unit are termed 5′ or 3′ flanking sequences. A functional genomic form of a gene typically contains regulatory elements necessary, and sometimes sufficient, for the regulation of transcription. The term “promoter” is generally used to describe a DNA region, typically but not exclusively 5′ of the site of transcription initiation, sufficient to confer accurate transcription initiation. In some aspects, a “promoter” also includes other cis-acting regulatory elements that are necessary for strong or elevated levels of transcription, or confer inducible transcription. In some embodiments, a promoter is constitutively active, while in alternative embodiments, the promoter is conditionally active (e.g., where transcription is initiated only under certain physiological conditions).

Generally, the term “regulatory element” refers to any cis-acting genetic element that controls some aspect of the expression of nucleic acid sequences. In some uses, the term “promoter” comprises essentially the minimal sequences required to initiate transcription. In some uses, the term “promoter” includes the sequences to start transcription, and in addition, also include sequences that can upregulate or downregulate transcription, commonly termed “enhancer elements” and “repressor elements”, respectively.

Specific DNA regulatory elements, including promoters and enhancers, generally only function within a class of organisms. For example, regulatory elements from the bacterial genome generally do not function in eukaryotic organisms. However, regulatory elements from more closely related organisms frequently show cross functionality. For example, DNA regulatory elements from a particular mammalian organism, such as human, will most often function in other mammalian species, such as mouse. Furthermore, in designing recombinant genes that will function across many species, there are consensus sequences for many types of regulatory elements that are known to function across species, e.g., in all mammalian cells, including mouse host cells and human host cells.

As used herein, the term “genome” refers to the total genetic information or hereditary material possessed by an organism (including viruses), i.e., the entire genetic complement of an organism or virus. The genome generally refers to all of the genetic material in an organism's chromosome (s), and in addition, extra-chromosomal genetic information that is stably transmitted to daughter cells (e.g., the mitochondrial genome). A genome can comprise RNA or DNA. A genome can be linear (mammals) or circular (bacterial). The genomic material typically resides on discrete units such as the chromosomes.

As used herein, the terms “vector”, “vehicle”, “construct” and “plasmid” are used in reference to any recombinant polynucleotide molecule that can be propagated and used to transfer nucleic acid segment (s) from one organism to another. Vectors generally comprise parts which mediate vector propagation and manipulation (e.g., one or more origin of replication, genes imparting drug or antibiotic resistance, a multiple cloning site, operably linked promoter/enhancer elements which enable the expression of a cloned gene, etc.). Vectors are generally recombinant nucleic acid molecules, often derived from bacteriophages, or plant or animal viruses. Plasmids and cosmids refer to two such recombinant vectors. A “cloning vector” or “shuttle vector” or “subcloning vector” contains operably linked parts that facilitate subcloning steps (e.g., a multiple cloning site containing multiple restriction endonuclease target sequences). A nucleic acid vector can be a linear molecule, or in circular form, depending on type of vector or type of application. Some circular nucleic acid vectors can be intentionally linearized prior to delivery into a cell.

As used herein, the term “expression vector” refers to a recombinant vector comprising operably linked polynucleotide elements that facilitate and optimize expression of a desired gene (e.g., a gene that encodes a protein) in a particular host organism (e.g., a bacterial expression vector or mammalian expression vector). Polynucleotide sequences that facilitate gene expression can include, for example, promoters, enhancers, transcription termination sequences, and ribosome binding sites.

As used herein, the term “host cell” refers to any cell that contains a heterologous nucleic acid. The heterologous nucleic acid can be a vector, such as a shuttle vector or an expression vector. In some aspects, the host cell is able to drive the expression of genes that are encoded on the vector. In some aspects, the host cell supports the replication and propagation of the vector. Host cells can be bacterial cells such as E. coli, or mammalian cells (e.g., human cells or mouse cells). When a suitable host cell (such as a suitable mouse cell) is used to create a stably integrated cell line, that cell line can be used to create a complete transgenic organism.

Methods (i.e., means) for delivering vectors/constructs or other nucleic acids (such as in vitro transcribed RNA) into host cells such as bacterial cells and mammalian cells are well known to one of ordinary skill in the art, and are not provided in detail herein. Any method for nucleic acid delivery into a host cell finds use with the invention.

For example, methods for delivering vectors or other nucleic acid molecules into bacterial cells (termed transformation) such as Escherichia coli are routine, and include electroporation methods and transformation of E. coli cells that have been rendered competent by previous treatment with divalent cations such as CaCl₂.

Methods for delivering vectors or other nucleic acid (such as RNA) into mammalian cells in culture (termed transfection) are routine, and a number of transfection methods find use with the invention. These include but are not limited to calcium phosphate precipitation, electroporation, lipid-based methods (liposomes or lipoplexes) such as Transfectamine® (Life Technologies™) and TransFectin™ (Bio-Rad Laboratories), cationic polymer transfections, for example using DEAE-dextran, direct nucleic acid injection, biolistic particle injection, and viral transduction using engineered viral carriers (termed transduction, using e.g., engineered herpes simplex virus, adenovirus, adeno-associated virus, vaccinia virus, Sindbis virus), and sonoporation. Any of these methods find use with the invention.

The invention farther provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell is preferably a prokaryotic cell, e.g., a DH5a cell. For purposes of producing a recombinant modified TCR, polypeptide, or protein, the host cell is preferably a mammalian cell. Most preferably, the host cell is a human cell. The host cell can be of any cell type, can originate from any type of tissue, and can be of any developmental stage.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

As used herein, the term “recombinant” in reference to a nucleic acid or polypeptide indicates that the material (e.g., a recombinant nucleic acid, gene, polynucleotide, polypeptide, etc.) has been altered by human intervention. Generally, the arrangement of parts of a recombinant molecule is not a native configuration, or the primary sequence of the recombinant polynucleotide or polypeptide has in some way been manipulated. A naturally occurring nucleotide sequence becomes a recombinant polynucleotide if it is removed from the native location from which it originated (e.g., a chromosome), or if it is transcribed from a recombinant DNA construct. A gene open reading frame is a recombinant molecule if that nucleotide sequence has been removed from it natural context and cloned into any type of nucleic acid vector (even if that ORF has the same nucleotide sequence as the naturally occurring gene). Protocols and reagents to produce recombinant molecules, especially recombinant nucleic acids, are well known to one of ordinary skill in the art. In some embodiments, the term “recombinant cell line” refers to any cell line containing a recombinant nucleic acid, that is to say, a nucleic acid that is not native to that host cell.

As used herein, the term “marker” most generally refers to a biological feature or trait that, when present in a cell (e.g., is expressed), results in an attribute or phenotype that visualizes or identifies the cell as containing that marker. A variety of marker types are commonly used, and can be for example, visual markers such as color development, e.g., lacZ complementation (β-galactosidase) or fluorescence, e.g., such as expression of green fluorescent protein (GFP) or GFP fusion proteins, RFP, BFP, selectable markers, phenotypic markers (growth rate, cell morphology, colony color or colony morphology, temperature sensitivity), auxotrophic markers (growth requirements), antibiotic sensitivities and resistances, molecular markers such as biomolecules that are distinguishable by antigenic sensitivity (e.g., blood group antigens and histocompatibility markers), cell surface markers (for example H2KK), enzymatic markers, and nucleic acid markers, for example, restriction fragment length polymorphisms (RFLP), single nucleotide polymorphism (SNP) and various other amplifiable genetic polymorphisms.

As used herein, the expressions “selectable marker” or “screening marker” or “positive selection marker” refer to a marker that, when present in a cell, results in an attribute or phenotype that allows selection or segregated of those cells from other cells that do not express the selectable marker trait. A variety of genes are used as selectable markers, e.g., genes encoding drug resistance or auxotrophic rescue are widely known. For example, kanamycin (neomycin) resistance can be used as a trait to select bacteria that have taken up a plasmid carrying a gene encoding for bacterial kanamycin resistance (e.g., the enzyme neomycin phosphotransferase II). Non-transfected cells will eventually die off when the culture is treated with neomycin or similar antibiotic.

A similar mechanism can also be used to select for transfected mammalian cells containing a vector carrying a gene encoding for neomycin resistance (either one of two aminoglycoside phosphotransferase genes; the neo selectable marker). This selection process can be used to establish stably transfected mammalian cell lines.

As used herein, the term “reporter” refers generally to a moiety, chemical compound or other component that can be used to visualize, quantitate or identify desired components of a system of interest. Reporters are commonly, but not exclusively, genes that encode reporter proteins. For example, a “reporter gene” is a gene that, when expressed in a cell, allows visualization or identification of that cell, or permits quantitation of expression of a recombinant gene. For example, a reporter gene can encode a protein, for example, an enzyme whose activity can be quantitated, for example, chloramphenicol acetyltransferase (CAT) or firefly luciferase protein. Reporters also include fluorescent proteins, for example, green fluorescent protein (GFP) or any of the recombinant variants of GFP, including enhanced GFP (EGFP), blue fluorescent proteins (BFP and derivatives), cyan fluorescent protein (CFP and other derivatives), yellow fluorescent protein (YFP and other derivatives) and red fluorescent protein (RFP and other derivatives).

As used herein, the terms “bacteria” or “bacterial” refer to prokaryotic Eubacteria, and are distinguishable from Archaea, based on a number of well-defined morphological and biochemical criteria.

As used herein, the term “eukaryote” refers to organisms (typically multicellular organisms) belonging to the Kingdom Eucarya, generally distinguishable from prokaryotes by the presence of a membrane-bound nucleus and other membrane-bound organelles, linear genetic material (i.e., linear chromosomes), the absence of operons, the presence of introns, message capping and poly-A mRNA, a distinguishing ribosomal structure and other biochemical characteristics.

As used herein, the terms “mammal” or “mammalian” refer to a group of eukaryotic organisms that are endothermic amniotes distinguishable from reptiles and birds by the possession of hair, three middle ear bones, mammary glands in females, a brain neocortex, and most giving birth to live young. The largest group of mammals, the placentals (Eutheria), have a placenta which feeds the offspring during pregnancy. The placentals include the orders Rodentia (including mice and rats) and primates (including humans).

As used herein, the term “encode” refers broadly to any process whereby the information in a polymeric macro-molecule is used to direct the production of a second molecule that is different from the first. The second molecule may have a chemical structure that is different from the chemical nature of the first molecule.

For example, in some aspects, the term “encode” describes the process of semi-conservative DNA replication, where one strand of a double-stranded DNA molecule is used as a template to encode a newly synthesized complementary sister strand by a DNA-dependent DNA polymerase. In other aspects, a DNA molecule can encode an RNA molecule (e.g., by the process of transcription that uses a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation. For example, the term “encode” refers to the capacity of a nucleic acid to provide another nucleic acid or a polypeptide. A nucleic acid sequence or construct is said to “encode” a polypeptide if it can be transcribed and/or translated to produce the polypeptide.

As used herein, the term “transcriptional element” is meant a region of DNA that can be transcribed that can be operably linked to a promoter in the vector or put into functional proximity with a promoter upon integration in the genome. In some cases, where the promoter and region of DNA to be transcribed are together in the transcriptional unit, the unit may be referred to as a “cassette”, for example the kanamycin/neomycin resistance cassette. The transcriptional unit can contain regions of DNA that are transcribed to produce mRNAs or regulatory RNAs, with or without promoter sequences.

As used herein, the term “target” or “targeting sequence” is not limited by the source of target DNA, which can be any source of DNA for which recombination is desired. For example, the target DNA can be located in a chromosome (i.e., genomic DNA) or can be in a vector, such as from a library.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

As used herein, the term “PiggyBac” or “PB” refers to a PiggyBac transposon and/or PiggyBac transposase that provides for a similar or increased frequency of transposition relative to a wild-type PiggyBac transposon and/or transposase.

As used herein, the term “PiggyBac transposase” or “PB transposase”, refers to the transposase isolated from the Trichoplusia ni (cabbage looper moth), or the nucleic acid sequence encoding said transposase.

As used herein, the term “operably linked”, refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration and/or excision functions (i.e., transposon sequences, transposase-encoding sequences, site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences), and sequences that provide replication functions (i.e., bacterial origins of replication, autonomous replication sequences, centromeric sequences).

As used herein, the term “gene products”, refers to either an RNA molecule or to a polypeptide resulting from the expression of a DNA sequence encoding for the RNA molecule or polypeptide.

As used herein, the term “recombinant expression vector” means a genetically-modified recombinant oligonucleotide or polynucleotide, which comprises nucleotide sequence encoding mRNA, protein, polypeptide, and peptide when the recombinant vector is contacted with the host cell under conditions sufficient to have the mRNA, protein, polypeptide or peptide expressed within the cell. The invention recombinant expression vector can comprise any type of nucleotides, including, but not limited to DNA and RNA, which can be single-stranded or double-stranded, synthesized or obtained in part from natural sources, and which can contain natural, non-natural or altered nucleotides. The bond between nucleotide can be naturally-occurring, and can also be non-naturally-occurring or modified.

The invention further provides any recombinant expression vector containing the inventive polynucleotide. The recombinant expression vector of the invention can be any suitable recombinant expression vector, and can be used to transform or transfect any suitable host. Suitable vectors include those designed for propagation and expansion or for expression or both, such as plasmids and viruses. The vector can be selected from the group consisting of the pUC series, the pcDNA series, the pBluescript series, the pET series, the pGEX series, and the pEX series. Bacteriophage vectors, such as λGT10, λGTI11, λZapII, λEMBL4, etc. also can be used. Examples of plant expression vectors include pBI01, pBI101.2, pBI101.3, pBI121 and pBIN19. Examples of animal expression vectors include pEUK-Cl, pMAM and pMAMneo. Preferably, the recombinant expression vector is pcDNA series.

The recombinant expression vectors of the invention can be prepared using standard recombinant DNA techniques. Constructs of expression vectors, which are circular or linear, can be prepared to contain a replication system functional in a prokaryotic or eukaryotic host cell. Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or normative promoter. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus. The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. The term “suicide gene” refers to a gene that causes the cell expressing the suicide gene to die. The suicide gene can be a gene that confers sensitivity to an agent, e.g., a drug, upon the cell in which the gene is expressed, and causes the cell to die. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

In the present, the eukaryotic cells can be any kind of cells such as a T cell, a B cell, a macrophage, a neutrophil, an erythrocyte, a hepatocyte, an endothelial cell, an epithelial cell, a muscle cell, a brain cell, etc. And the tissues or organisms can be any kind of the non-reproductive tissues such as liver, lungs, heart, brain, eye, stomach, pancreas, spleen, bladder, etc.

### EXAMPLES

### Example 1: Plasmids Construction

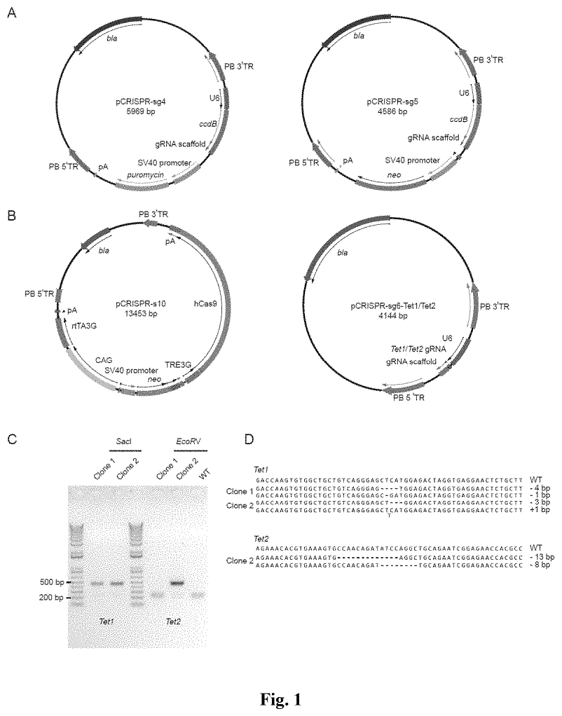

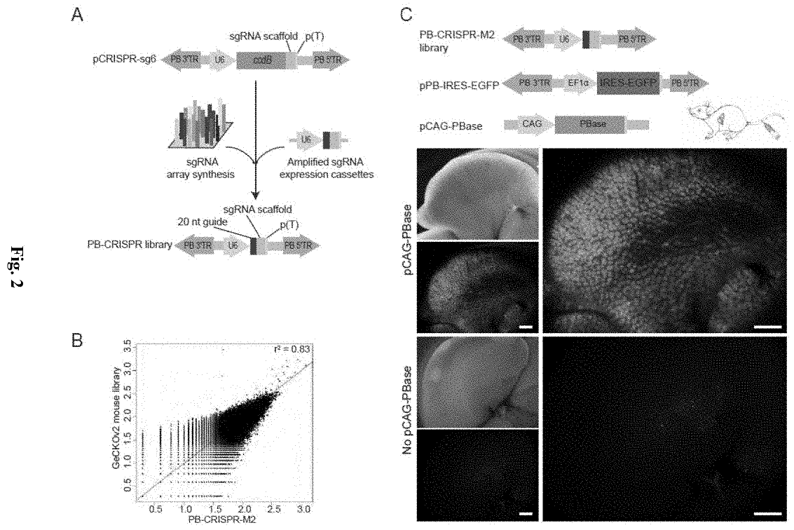

To utilize PB to deliver and express a genome-wide single guide RNA (sgRNA) library for high-throughput screening, we constructed three PB vectors, pCRISPR-sg4, pCRISPR-sg5 and pCRISPR-sg6, which all express an sgRNA under control of the human U6 promoter. pCRISPR-sg4 and pCRISPR-sg5 and pCRISPR-sg6 were constructed by PCR assembly of the U6-sgRNA expression cassette from pX330 (Cong, L. et al. Science 339, 819-823 (2013)), SV40-neo from pIRES2-EGFP (Clontech), puro from pMSCVpuro (BD biosciences), and ccdB from pStart-K (Wu, S., Ying, G, Wu, Q. & Capecchi, M. R. Nat. Protoc. 3, 1056-1076 (2008)) on a PB backbone from pZGs (Wu, S., Ying, G, Wu, Q. & Capecchi, M. R. Nat. Genet. 39, 922-930 (2007)). pCRISPR-sg4 and pCRISPR-sg5 carry puromycin and neo resistance genes respectively (FIG. 1a), enabling convenient use in cultured cells. PB vectors tend to have multiple copy integrations for inserts<10 kb, and single copy integration for inserts >10 kb (Woltjen, K. et al. Nature 458, 766-770 (2009); Li, M. A. et al. Nucleic Acids Res 39, 9 (2011)). To make PB more efficient for in vivo uses, pCRISPR-sg6 was designed to contain minimal sgRNA expression elements without any selectable marker and associated promoter, thus more likely to result in multiple copy insertions. The inclusion of the toxic gene ccdB in these vectors ensures that essentially no background colonies can grow during library construction (FIG. 2a).

pPB-hNRAS^{G12V} was constructed by PCR assembly of NRAS^{G12V} amplified from cDNA, and IRES-EGFP from pIRES2-EGFP on a PB backbone from pZGs (Wu, S., Ying, G, Wu, Q. & Capecchi, M. R. Nat. Genet. 39, 922-930 (2007)).

To construct the pCRISPR-W9 backbone, PB terminal repeats were amplified from pZGs (Wu, S., Ying, G, Wu, Q. & Capecchi, M. R. Nat. Genet. 39, 922-930 (2007)) and inserted into pX330 (Cong, L. et al. Science 339, 819-823 (2013)), and GFP was added to Cas9 gene with a 2A sequence.

sgRNA targeting individual genes was PCR amplified from oligonucleotide template with primers xc1732/xc1733 (Table 1). The purified PCR products were cloned into the BbsI site of pCRISPR-sg6 using the Gibson Assembly method (NEB), resulting in pCRISPR-sg6-Trp53, and pCRISPR-sg6-Cdkn2b plasmids. All plasmids were confirmed by sequencing. Qiagen EndoFree Plasmid Maxi Kit was used to prepare plasmid DNA for injection.

TABLE 1
Primers used in this study

| SEQ | | | |
|---|---|---|---|
| ID NO | Primer Name | Primer sequence | Note |
| 1 | Non_lib Cdkn2a | ATATCTTGTGGAAAGGACGAA | Construction of pCRISPR-W9- |
| | | ACACCGCGGTGCAGATTCGAA | Cdkn2a-sgRNA |
| | | CTGCGGTTTTAGAGCTAGAAAT | |
| | | AGCAAGTTAA | |
| 2 | A_56035_Trp53 | ATATCTTGTGGAAAGGACGAA | Construction of pCRISPR-sg6-Trp53 |
| | | ACACCGTGAGGGCTTACCATC | |
| | | ACCATGTTTTAGAGCTAGAAAT | |
| | | AGCAAGTTAA | |
| 3 | B_09614_Cdkn2b | ATATCTTGTGGAAAGGACGAA | Construction of pCRISPR-sg6-Cdkn2b |
| | | ACACCGGCAGCACGACAAGCG | |
| | | TGTCCGTTTTAGAGCTAGAAAT | |
| | | AGCAAGTTAA | |
| 4 | Tet1-gRNA-F | CACCGGCTGCTGTCAGGGAGC | Amplification of Tet1 target site of |
| | | TCA | Tet1-gRNA |
| 5 | Tet1-gRNA-R | AAACTGAGCTCCCTGACAGCA | Amplification of Tet1 target site of |
| | | GCC | Tet1-gRNA |
| 6 | Tet2-gRNA-F | CACCGAAAGTGCCAACAGATA | Amplification of Tet2 target site of |
| | | TCC | Tet2-gRNA |
| 7 | Tet2-gRNA-R | AAACGGATATCTGTTGGCACTT | Amplification of Tet2 target site of |
| | | TC | Tet2-gRNA |
| 8 | Qcas9-F | CCGAAGAGGTCGTGAAGAAG | Quantitative RT-PCR analysis for Cas9 |
| | | | expression |
| 9 | Qcas9-R | GCCTTATCCAGTTCGCTCAG | Quantitative RT-PCR analysis for Cas9 |
| | | | expression |
| 10 | QhNRAS-F | ACAGTGCCATGAGAGACCAA | Quantitative RT-PCR analysis for |
| | | | hNRAS expression |
| 11 | QhNRAS-R | CTCGCTTAATCTGCTCCCTGT | Quantitative RT-PCR analysis for |
| | | | hNRAS expression |
| 12 | QmGADPH-F | cttcaacagcaactcccactc | Quantitative RT-PCR analysis for |
| | | | mGADPH expression |
| 13 | QmGADPH-R | cctgttgctgtagccgtattc | Quantitative RT-PCR analysis for |
| | | | mGADPH expression |
| 14 | xcl732 | ATATCTTGTGGAAAGGACGAA | Construction of sgRNA plasmids |
| | | ACACCG | |
| 15 | xcl733 | TTAACTTGCTATTTCTAGCTCTA | Construction of sgRNA plasmids |
| | | AAAC | |
| 16 | Tet1-gRNA-F | CACCGGCTGCTGTCAGGGAGC | Amplification of Tet1 target site of |
| | | TCA | Tet1-gRNA |
| 17 | Tet1-gRNA-R | AAACTGAGCTCCCTGACAGCA | Amplification of Tet1 target site of |
| | | GCC | Tet1-gRNA |
| 18 | Tet2-gRNA-F | CACCGAAAGTGCCAACAGATA | Amplification of Tet2 target site of |
| | | TCC | Tet2-gRNA |
| 19 | Tet2-gRNA-R | AAACGGATATCTGTTGGCACTT | Amplification of Tet2 target site of |
| | | TC | Tet2-gRNA |
| 20 | Non_lib Cdkn2a | GTCAGAAGCTTTTGGACCAAC | Amplification of Cdkn2a target site of |
| | target-F | T | Non_lib Cdkn2a |
| 21 | Non_lib Cdkn2a | ACAATCCCAGTTCGGCTTAAA | Amplification of Cdkn2a target site of |
| | target-R | G | Non_lib Cdkn2a |
| 22 | A_54001_Tle4 | ATATCGAAAGTTTGGCCTCAGC | Amplification of Tle4 target site of |
| | target-F | GT | sgRNA_A54001 |
| 23 | A_54001_Tle4 | ACGGGCCACTTTCGATTCCGG | Amplification of Tle4 target site of |
| | target-R | GTA | sgRNA_A54001 |
| 24 | A_36261_Olfr1311 | TGTGGTCTCATGATGGTCAAGT | Amplification of Olfr1311 target site of |
| | target-F | AG | sgRNA_A36261 |
| 25 | A_36261_Olfr1311 | TGGCTCATGGTATTGAATGGCT | Amplification of Olfr1311 target site of |
| | target-R | GA | sgRNA_A36261 |
| 26 | B_28812_Lgals7 | TCTTGGGTTTCACCAGCACGTC | Amplification of Lgals7 target site of |
| | target-F | CT | sgRNA_B28812 |
| 27 | B_28812_Lgals7 | TCCAGGGTAGCTTCAAGATCC | Amplification of Lgals7 target site of |
| | target-R | AA | sgRNA_B28812 |
| 28 | A_28412_Lamc2 | AGCAACTTCATGGTGGCTCAC | Amplification of Lamc2 target site of |
| | target-F | AAC | sgRNA_A28412 |
| 29 | A_28412_Lamc2 | TTCACCCCCTTCTTTCTGTGGA | Amplification of Lamc2 target site of |
| | target-R | GC | sgRNA_A28412 |
| 30 | B_27405_Kif5a | ACATTGTCCCTCACTTCATCCT | Amplification of Kif5a target site of |
| | target-F | CCA | sgRNA_B27405 |
| 31 | B_27405_Kif5a | AGCCTAAGTATGTGACACGCCT | Amplification of Kif5a target site of |
| | target-R | TT | sgRNA_B27405 |
| 32 | B_51283_Srms | AGGAATGGAGGGGAGGAAGG | Amplification of Srms target site of |
| | target-F | AAGA | sgRNA_B51283 |
| 33 | B_51283_Srms | TGAGGCCTGGTAGTTATGTTAG | Amplification of Srms target site of |
| | target-R | AG | sgRNA_B51283 |
| 34 | A_07138_Brap | TTGTGTGGGTTGAGTGCCGTAC | Amplification of Brap target site of |
| | target-F | TG | sgRNA_A07138 |
| 35 | A_07138_Brap | ATACACATGATCCCACCACTCA | Amplification of Brap target site of |
| | target-R | GG | sgRNA_A07138 |
| 36 | A_26940_Kcnh1 | GTGAGTGCTGATGAGATTTTCA | Amplification of Kcnh1 target site of |
| | target-F | AG | sgRNA_A26940 |
| 37 | A_26940_Kcnh1 | TCCAAGTGTAACTATGGAATGG | Amplification of Kcnh1 target site of |
| | target-R | TG | sgRNA_A26940 |
| 38 | B_25175_Hyal5 | AAGATTGGGAAAGTCACTTCG | Amplification of Hyal5 target site of |
| | target-F | GCC | sgRNA_B25175 |
| 39 | B_25175_Hyal5 | GCAAACTTCAAGTCCTAGCAA | Amplification of Hyal5 target site of |
| | target-R | CAG | sgRNA_B25175 |
| 40 | B_21517_Gm4981 | TTTGCTCCCCTGTTTCCTCCAC | Amplification of Gm4981 target site of |
| | target-F | AT | sgRNA_B21517 |
| 41 | B_21517_Gm4981 | ACAGCTCAAGATCAAGACTTG | Amplification of Gm4981 target site of |
| | target-R | CTG | sgRNA_B21517 |
| 42 | A_02125_AK129341 | ACAGTTTCCCCTCTTGCATCTC | Amplification of AK129341 target site |
| | target-F | GT | of sgRNA_A02125 |
| 43 | A_02125_AK129341 | GAGACCATGAAAGCAAATACC | Amplification of AK129341 target site |
| | target-R | GAG | of sgRNA_A02125 |
| 44 | A_63598_mmu-mir- | TATGGGGTGGAGGAGAACTGT | Amplification of mmu-mir-99b target |
| | 99b target-F | GAG | site of sgRNA_A63598 |
| 45 | A_63598_mmu-mir- | GCTCCTATCAAGAACTCTTGGG | Amplification of mmu-mir-99b target |
| | 99b target-R | CA | site of sgRNA_A63598 |
| 46 | A_08684_Ccdc87 | TGCGCAGGCGCATTGATGCAG | Amplification of Ccdc87 target site of |
| | target-F | TTT | sgRNA_A08684 |
| 47 | A_08684_Ccdc87 | GATAGACATCAGGACTGGTGA | Amplification of Ccdc87 target site of |
| | target-R | GGA | sgRNA_A08684 |
| 48 | B_34017_Ninj2 | TTCATTTCTTCCTGATCGGTCTC | Amplification of Ninj2 target site of |
| | target-F | C | sgRNA_B34017 |
| 49 | B_34017_Ninj2 | ATAACCTAGCATTCAAGGTGCA | Amplification of Ninj2 target site of |
| | target-R | GA | sgRNA_B34017 |
| 50 | A_30543_Mbd6 | AACTCACCAGGAGAAGAGTGT | Amplification of Mbd6 target site of |
| | target-F | GAG | sgRNA_A30543 |
| 51 | A_30543_Mbd6 | TGCTGTGTACTATATCAGGTATG | Amplification of Mbd6 target site of |
| | target-R | GC | sgRNA_A30543 |
| 52 | B_01105_4430402I18Rik | AGGAGCGTTCTAGGACATCAT | Amplification of 4430402I18Rik target |
| | target-F | GTG | site of sgRNA_B01105 |
| 53 | B_01105_4430402I18Rik | CTGACATAAGCAACCTCAGGA | Amplification of 4430402I18Rik target |
| | target-R | ATG | site of sgRNA_B01105 |
| 54 | B_53696_Thbs2 | AACACTGAGACAGCTCAGTTC | Amplification of Thbs2 target site of |
| | target-F | CCA | sgRNA_B53696 |
| 55 | B_53696_Thbs2 | TGAGCTCCGCAGTACAGTCTTT | Amplification of Thbs2 target site of |
| | target-R | GT | sgRNA_B53696 |
| 56 | A_28398_Lama5 | TAGGTAGATGAGGACAGACAG | Amplification of Lama5 target site of |
| | target-F | ACA | sgRNA_A28398 |
| 57 | A_28398_Lama5 | TGCAGCCTCCAAGAGGGATTG | Amplification of Lama5 target site of |
| | target-R | TTT | sgRNA_A28398 |
| 58 | B_19120_Fxyd4 | CTTACCATAGTAGAAGGGACTG | Amplification of Fxyd4 target site of |
| | target-F | TC | sgRNA_B19120 |
| 59 | B_19120_Fxyd4 | ATCTGGTAGGCCTAGGATCAGG | Amplification of Fxyd4 target site of |
| | target-R | GT | sgRNA_B19120 |
| 60 | A_36051_Olfr1239 | AAGAGTGACTCCCTTTCTTAGT | Amplification of Olfr1239 target site of |
| | target-F | GC | sgRNA_A36051 |
| 61 | A_36051_Olfr1239 | TATAACCTTCCTTTCTGTGGTC | Amplification of Olfr1239 target site of |
| | target-R | CT | sgRNA_A36051 |
| 62 | A_45045_Reep5 | TGCATGGAGATTAACCTGGGTC | Amplification of Reep5 target site of |
| | target-F | AA | sgRNA_A45045 |
| 63 | A_45045_Reep5 | AACCAGCAGCAACAAGAAAC | Amplification of Reep5 target site of |
| | target-R | ACCC | sgRNA_A45045 |
| 64 | B_10774_Clec5a | ATCAGCTATCTCAGGTATCTCA | Amplification of Clec5a target site of |
| | target-F | GG | sgRNA_B10774 |
| 65 | B_10774_Clec5a | TTCCTGATTCGCAGAACCAGA | Amplification of Clec5a target site of |
| | target-R | CCA | sgRNA_B10774 |
| 66 | A_63693_mmu-mir- | AGGGGATAGAACTTATGTGGA | Amplification of mmu-mir-6970 target |
| | 6970 target-F | GGT | site of sgRNA_A63693 |
| 67 | A_63693_mmu-mir- | TGAATTGGTGGGATCAGAAGT | Amplification of mmu-mir-6970 target |
| | 6970 target-R | GGA | site of sgRNA_A63693 |
| 68 | B_21757_Gm5941 | ATGGTAGGCACCTGGAAGTTC | Amplification of Gm5941 target site of |
| | target-F | AAC | sgRNA_B21757 |
| 69 | B_21757_Gm5941 | ATCTCCCTCAACCAGAGTGATC | Amplification of Gm5941 target site of |
| | target-R | TC | sgRNA_B21757 |
| 70 | A_36065_Olfr1243 | TCCAGCTACCAGCAACAGAAG | Amplification of Olfr1243 target site of |
| | target-F | AAT | sgRNA_A36065 |
| 71 | A_36065_Olfr1243 | CCAAGGAAGAGTAGACATCAA | Amplification of Olfr1243 target site of |
| | target-R | CCT | sgRNA_A36065 |
| 72 | B_17813_Fastkd5 | ACGAGTGCCCTTCAGAGAGCA | Amplification of Fastkd5 target site of |
| | target-F | GAG | sgRNA_B17813 |
| 73 | B_17813_Fastkd5 | TGACTTAGAGGTTCAGCTTGAT | Amplification of Fastkd5 target site of |
| | target-R | GC | sgRNA_B17813 |
| 74 | B_09614_Cdkn2b | TACTAAATCTCCTTGGTGATCC | Amplification of Cdkn2b target site of |
| | target-F | CC | sgRNA_B09614 |
| 75 | B_09614_Cdkn2b | TTTCTTATTGCTTCACCTGTGG | Amplification of Cdkn2b target site of |
| | target-R | AG | sgRNA_B09614 |
| 76 | B_41633_Pml | AGGACCTTGGTGTCTCTTTAGG | Amplification of Pml target site of |
| | target-F | AC | sgRNA_B41633 |
| 77 | B_41633_Pml | CCGGATCTTTCCTTGTTCTGCT | Amplification of Pml target site of |
| | target-R | AA | sgRNA_B41633 |
| 78 | A_53331_Tecr | AGAGGCAACAAGCCGATGAGG | Amplification of Tecr target site of |
| | target-F | GAA | sgRNA_A53331 |
| 79 | A_53331_Tecr | TAGCTTGTTCCTGACCTGCCTG | Amplification of Tecr target site of |
| | target-R | TA | sgRNA_A53331 |
| 80 | A_35899_Olfr1181 | AGGTTGAAAGAGCTTTGCGTC | Amplification of Olfr1181 target site of |
| | target-F | TCC | sgRNA_A35899 |
| 81 | A_35899_Olfr1181 | GATGCAGTTCTCTGTTCAACCA | Amplification of Olfr1181 target site of |
| | target-R | AC | sgRNA_A35899 |
| 82 | A_55795_Trim36 | AGTAACCTATATGTAGTCCCAT | Amplification of Trim36 target site of |
| | target-F | CC | sgRNA_A55795 |
| 83 | A_55795_Trim36 | TGACCCTGTGTTGGTTTTCATC | Amplification of Trim36 target site of |
| | target-R | CT | sgRNA_A55795 |
| 84 | B_22324_Golga7 | AACAGTCCAGAGACCCAGACA | Amplification of Golga7 target site of |
| | target-F1 | ATG | sgRNA_B22324 |
| 85 | B_22324_Golga7 | TGTACAGCTGATAACTGTGTCC | Amplification of Golga7 target site of |
| | target-R1 | TG | sgRNA_B22324 |
| 86 | B_22324_Golga7 | ATTGGGAGACAAAGTGGATGC | Amplification of Golga7 target site of |
| | target-F2 | TGA | sgRNA_B22324 |
| 87 | B_22324_Golga7 | TGTTCATTAAGACTACAGCAGT | Amplification of Golga7 target site of |
| | target-R2 | GG | sgRNA_B22324 |
| 88 | B_55404_Tpd52 | AAGAAGTCAGGCAAGCACTTC | Amplification of Tpd52 target site of |
| | target-F | AGG | sgRNA_B55404 |
| 89 | B_55404_Tpd52 | AACACTTGAGTTTTGCCAGCC | Amplification of Tpd52 target site of |
| | target-R | CCA | sgRNA_B55404 |
| 90 | B_41976_Pon2 | TGGAGAAACCCAGAGACCTTT | Amplification of Pon2 target site of |
| | Target-F | ATC | sgRNA_B41976 |
| 91 | B_41976_Pon2 | ACCCACAATTCAAGAGTACAG | Amplification of Pon2 target site of |
| | Target-R | TGG | sgRNA_B41976 |
| 92 | A_53332_Tecr | AGAGGCAACAAGCCGATGAGG | Amplification of Tecr target site of |
| | target-F | GAA | sgRNA_A53332 |
| 93 | A_53332_Tecr | TAGCTTGTTCCTGACCTGCCTG | Amplification of Tecr target site of |
| | target-R | TA | sgRNA_A53332 |
| 94 | A_47925_Serpinb9c | TGAGGGACTTAAAAGTCTTTC | Amplification of Serpinb9c target |
| | target-R | ACC | site of sgRNA_A47925 |
| 95 | B_44752_Rbm15 | CGAATGGTGCCAAATCGGTCA | Amplification of Rbm15 target site of |
| | target-F | AA | sgRNA_B44752 |
| 96 | B_44752_Rbm15 | TGCTGCTCTGGGATACAGAGA | Amplification of Rbm15 target site of |
| | target-R | CTA | sgRNA_B44752 |
| 97 | A_12850_Cyp2g1 | TTTCCAAACCAGGTTGCAGTTT | Amplification of Gyp2g1 target site of |
| | target-F | GG | sgRNA_A12850 |
| 98 | A_12850_Cyp2g1 | AAGGCCAGCCTGAGCTACACA | Amplification of Gyp2g1 target site of |
| | target-R | AAG | sgRNA_A12850 |
| 99 | A_39356_Paip2b | ATAAGCCTCTGGCTGCTAAGGC | Amplification of Paip2b target site of |
| | target-F | CT | sgRNA_A39356 |
| 100 | A_39356_Paip2b | TGGGGAACAAGGTTTACATAG | Amplification of Paip2b target site of |
| | target-R | CAT | sgRNA_A39356 |
| 101 | B_23557_H2-Q2 | ACAGATCACTTCAAGTGTCCTG | Amplification of H2-Q2 target site of |
| | target-F | CT | sgRNA_B23557 |
| 102 | B_23557_H2-Q2 | CATGTTCCACATGGCATGTGTA | Amplification of H2-Q2 target site of |
| | target-R | TC | sgRNA_B23557 |
| 103 | B_16359_Epm2aip1 | AAATCTCCAGCCAATAGGAAC | Amplification of Epm2aip1 target |
| | target-F | GGA | site of sgRNA_B16359 |
| 104 | B_16359_Epm2aip1 | TGCACTGGTGTACGAAGTCAC | Amplification of Epm2aip1 target |
| | target-R | CCT | site of sgRNA_B16359 |
| 105 | B_19898_Gif | ATTACCTCTGAGCTGTACCACT | Amplification of Gif target site of |
| | target-F | CA | sgRNA_B19898 |
| 106 | B_19898_Gif | TGAAGTGTCATCAGAGGTAGC | Amplification of Gif target site of |
| | target-R | TCT | sgRNA_B19898 |
| 107 | B_31702_Morn1 | AACTCACTTTGTAGACCAGGC | Amplification of Morn1 target site of |
| | target-F | TGG | sgRNA_B31702 |
| 108 | A_09612_Cdkn2b | AGTGTTGGCTTCTTTCTATGAC | Amplification of Cdkn2b target site of |
| | target-F | TG | sgRNA_A09612 |
| 109 | A_09612_Cdkn2b | TGCAGAACGCTGCAGCTCAGT | Amplification of Cdkn2b target site of |
| | target-R | GCC | sgRNA_A09612 |
| 110 | A_19126_Fxyd4 | AGCCAAAGATCCGTACCACTT | Amplification of Fxyd4 target site of |
| | target-F | GGC | sgRNA_A19126 |
| ill | A_19126_Fxyd4 | TTCTGAATGAATGTGTGAGGGT | Amplification of Fxyd4 target site of |
| | target-R | AC | sgRNA_A19126 |
| 112 | B_31702_Morn1 | ACAGACAGACAAACATACATA | Amplification of Morn1 target site of |
| | target-R | CAG | sgRNA_B31702 |
| 113 | B_44494_Rap1gap2 | ACCTGAGGTCTCCACTAGCCT | Amplification of Rap1gap2 target |
| | target-F | GAT | site of sgRNA_B44494 |
| 114 | B_44494_Rap1gap2 | TGTTCCAGGTCACCAGTCTAG | Amplification of Rap1gap2 target |
| | target-R | GAAG | site of sgRNA_B44494 |
| 115 | B_57494_Usp14 | ATGCCACTCATCCAAAAGTCA | Amplification of Usp14 target site of |
| | target-F | ACC | sgRNA_B57494 |
| 116 | B_57494_Usp14 | TTTTGGCCAGGTGAATTGATAG | Amplification of Usp14 target site of |
| | target-R | GC | sgRNA_B57494 |
| 117 | A_33521_Ndufa11 | AATAAGACCTCGGTACAAACC | Amplification of Ndufa11 target site of |
| | target-F | TGC | sgRNA_A33521 |
| 118 | A_33521_Ndufa11 | TTCAAAAACTCCGATGACCCG | Amplification of Ndufa11 target site of |
| | target-R | ATC | sgRNA_A33521 |
| 119 | A_46638_Rspry1 | GTCCACTTTAGGACTATGAACA | Amplification of Rspry1 target site of |
| | target-F | GC | sgRNA_A46638 |
| 120 | A_46638_Rspry1 | TTTACCCCCTCCGTGTTATGTG | Amplification of Rspry1 target site of |
| | target-R | TC | sgRNA_A46638 |
| 121 | A_57601_Usp38 | ATGTCTGACACTGAAGCAGAA | Amplification of Usp38 target site of |
| | target-F | CTG | sgRNA_A57601 |
| 122 | A_57601_Usp38 | AGCTTGCCAATTGAACAGTGTA | Amplification of Usp38 target site of |
| | target-R | TG | sgRNA_A57601 |
| 123 | B_06455_Baat | TACTCTCCTTCCTTGCCAGATA | Amplification of Baat target site of |
| | target-F | AG | sgRNA_B06455 |
| 124 | B_06455_Baat | TCTACCCACCTGTACCCAGTAA | Amplification of Baat target site of |
| | target-R | TG | sgRNA_B06455 |
| 125 | A_18990_Fsd11 | CATGAGAACTATTGGGTTGTGT | Amplification of Fsd11 target site of |
| | target-F | GG | sgRNA_A18990 |
| 126 | A_18990_Fsd11 | AACTGCATCCCAGCAGGGTAC | Amplification of Fsd11 target site of |
| | target-R | AT | sgRNA_A18990 |
| 127 | B_54358_Tmem151a | TCCACTTAAGCTTCGGAAGAC | Amplification of Tmem151a target site |
| | target-F | CCC | of sgRNA_B54358 |
| 128 | B_54358_Tmem151a | AAGTGCTTCAGCTTTGGGAGT | Amplification of Tmem151a target site |
| | target-R | GCT | of sgRNA_B54358 |
| 129 | A_58447_Vmn1r63 | ATGTACTGAGGACACAGGTGG | Amplification of Vmn1r63 target site |
| | target-F | AG | of sgRNA_A58447 |
| 130 | A_58447_Vmn1r63 | GTTGATATTCTGGATCAATGTC | Amplification of Vmn1r63 target site |
| | target-R | C | of sgRNA_A58447 |
| 131 | A_29995_Mael | AGAGTTTTGGGCTGCAAGTCC | Amplification of Mael target site of |
| | target-F | AGC | sgRNA_A29995 |
| 132 | A_29995_Mael | TAGCTATAGAAGTTGTTTGCCA | Amplification of Mael target site of |
| | target-R | TG | sgRNA_A29995 |
| 133 | B_49633_Slc6a14 | TGTACTCTGCAGACACCTGCTT | Amplification of Slc6a14 target site of |
| | target-F | TC | sgRNA_B49633 |
| 134 | B_49633_Slc6a14 | GTACTTCTCATTGTGGCCTTGA | Amplification of Slc6a14 target site of |
| | target-R | TC | sgRNA_B49633 |
| 135 | B_47599_Sel1l2 | GATGAACAAGATCAGCATCTAT | Amplification of Sel1l2 target site of |
| | target-F | AC | sgRNA_B47599 |
| 136 | B_47599_Sel1l2 | CACAGTGTCACCACAATGTTTC | Amplification of Sel1l2 target site of |
| | target-R | C | sgRNA_B47599 |
| 137 | A_44928_Rcbtb2 | ACGAGGCAGTTTGCTTTAGGA | Amplification of Rcbtb2 target site of |
| | target-F | AGG | sgRNA_A44928 |
| 138 | A_44928_Rcbtb2 | TGTCACGCAATGATTCCACTCT | Amplification of Rcbtb2 target site of |
| | target-R | GA | sgRNA_A44928 |
| 139 | B_15909_Elane | TGACCTCTGGTCCATCTCTTTC | Amplification of Elane target site of |
| | target-F | AT | sgRNA_B15909 |
| 140 | B_15909_Elane | AGCACTACCTGCACTGACCGG | Amplification of Elane target site of |
| | target-R | AAA | sgRNA_B15909 |
| 141 | B_01812_9130204L05Rik | AGACTTCAGAAGCATGGAGAG | Amplification of 9130204L05Rik target |
| | target-F | CAC | site of sgRNA_B01812 |
| 142 | B_01812_9130204L05Rik | CTGCAAAACAGAGTCCTAGCT | Amplification of 9130204L05Rik target |
| | target-R | CTG | site of sgRNA_B01812 |
| 143 | A_60072_Zbtb37 | TGGCCCAAGCCACTCTTCTAGA | Amplification of Zbtb37 target site of |
| | target-F | TT | sgRNA_A60072 |
| 144 | A_60072_Zbtb37 | TATTTCCGGGATCACATGTCCT | Amplification of Zbtb37 target site of |
| | target-R | TG | sgRNA_A60072 |
| 145 | A_28484_Larp6 | TGTCCCCTTGGTTTCTATACCTA | Amplification of Larp6 target site of |
| | target-F | C | sgRNA_A28484 |
| 146 | A_28484_Larp6 | AATTTGCTAGGCAGGCAGCCTA | Amplification of Larp6 target site of |
| | target-R | TG | sgRNA_A28484 |
| 147 | xcl801- | gtttctatca | Amplification of sgRNA regions for |
| | CRScloneDeSeq-F | gagcgaggcgTGAAAGTATTTCGAT | sequencing |
| | | TTCTTGG | |
| 148 | xcl802- | gtttctatca | Amplification of sgRNA regions for |
| | CRScloneDeSeq-R | gagcgaggcgGTTGATAACGGACTA | sequencing |
| | | GCCTTATT | |
| 149 | xcl803- | gtttctatca | Amplification of sgRNA regions for |
| | CRScloneDeSeq-F | ctatggtggcTGAAAGTATTTCGATT | sequencing |
| | | TCTTGG | |
| 150 | xcl804- | gtttctatca | Amplification of sgRNA regions for |
| | CRScloneDeSeq-R | ctatggtggcGTTGATAACGGACTA | sequencing |
| | | GCCTTATT | |
| 151 | xcl805- | gtttctatca | Amplification of sgRNA regions for |
| | CRScloneDeSeq-F | atgccagtttTGAAAGTATTTCGATT | sequencing |
| | | TCTTGG | |
| 152 | xcl806- | gtttctatca | Amplification of sgRNA regions for |
| | CRScloneDeSeq-R | atgccagtttGTTGATAACGGACTAG | sequencing |
| | | CCTTATT | |
| 153 | xcl807- | gtttctatca | Amplification of sgRNA regions for |
| | CRScloneDeSeq-F | gcgcccgacaTGAAAGTATTTCGAT | sequencing |
| | | TTCTTGG | |
| 154 | xcl808- | gtttctatca | Amplification of sgRNA regions for |
| | CRScloneDeSeq-R | gcgcccgacaGTTGATAACGGACTA | sequencing |
| | | GCCTTATT | |
| 155 | xcl817- | gtttctatca | Amplification of sgRNA regions for |
| | CRScloneDeSeq-F | tgatccgtagTGAAAGTATTTCGATT | sequencing |
| | | TCTTGG | |
| 156 | xcl818- | gtttctatca | Amplification of sgRNA regions for |
| | CRScloneDeSeq-R | tgatccgtagGTTGATAACGGACTA | sequencing |
| | | GCCTTATT | |
| 157 | xcl819- | gtttctatca | Amplification of sgRNA regions for |
| | CRScloneDeSeq-F | aaggtgccctTGAAAGTATTTCGATT | sequencing |
| | | TCTTGG | |
| 158 | xcl820- | gtttctatca | Amplification of sgRNA regions for |
| | CRScloneDeSeq-R | aaggtgccctGTTGATAACGGACTA | sequencing |
| | | GCCTTATT | |
| 159 | xcl827- | gtttctatca | Amplification of sgRNA regions for |
| | CRScloneDeSeq-F | ggggttgcatTGAAAGTATTTCGATT | sequencing |
| | | TCTTGG | |
| 160 | xcl828- | gtttctatca | Amplification of sgRNA regions for |
| | CRScloneDeSeq-R | ggggttgcatGTTGATAACGGACTA | sequencing |
| | | GCCTTATT | |
| 161 | xcl829- | gtttctatca | Amplification of sgRNA regions for |
| | CRScloneDeSeq-F | tttgaccgcgTGAAAGTATTTCGATT | sequencing |
| | | TCTTGG | |
| 162 | xcl830- | gtttctatca | Amplification of sgRNA regions for |
| | CRScloneDeSeq-R | tttgaccgcgGTTGATAACGGACTA | sequencing |
| | | GCCTTATT | |
| 163 | xcl831- | gtttctatca | Amplification of sgRNA regions for |
| | CRScloneDeSeq-F | cgtgagtctaTGAAAGTATTTCGATT | sequencing |
| | | TCTTGG | |
| 164 | xcl832- | gtttctatca | Amplification of sgRNA regions for |
| | CRScloneDeSeq-R | cgtgagtctaGTTGATAACGGACTA | sequencing |
| | | GCCTTATT | |
| 165 | xcl833- | gtttctatca | Amplification of sgRNA regions for |
| | CRScloneDeSeq-F | gggtgaaagcTGAAAGTATTTCGAT | sequencing |
| | | TTCTTGG | |
| 166 | xcl834- | gtttctatca | Amplification of sgRNA regions for |
| | CRScloneDeSeq-R | gggtgaaagcGTTGATAACGGACTA | sequencing |
| | | GCCTTATT | |
| 167 | A_56035_Trp53 | ATTCTGCCAGCTGGCGAAGAC | Amplification of Trp53 target site of |
| | target F | GTG | sgRNA_A56035 |
| 168 | A_56035_Trp53 | ACTCGGGATACAAATTTCCTTC | Amplification of Trp53 target site of |
| | target R | CA | sgRNA_A56035 |
| 169 | A_24528_Hmox2 | TGAGTCTTCTTAGTTTAGGGAT | Amplification of Hmox2 target site of |
| | target F | GG | sgRNA_A24528 |
| 170 | A_24528_Hmox2 | CAGTTGCTGCCTCCTAGTGTAC | Amplification of Hmox2 target site of |
| | target R | CT | sgRNA_A24528 |
| 171 | B_30072_Magel2 | AGCTGACACCGGGAGTCCTGA | Amplification of Magel2 target site of |
| | target F | TGG | sgRNA_B30072 |
| 172 | B_30072_Magel2 | TTGTAGGATCAAAGGCTGACC | Amplification of Magel2 target site of |
| | target R | CTG | sgRNA_B30072 |
| 173 | A_38994_Osbpl11 | AAGGAAAGTAGTGCTAGCCTT | Amplification of Osbpl11 target site of |
| | target F | TGC | sgRNA_A38994 |
| 174 | A_38994_Osbpl11 | AATCACTCTACCTCCCTGGCTC | Amplification of Osbpl11 target site of |
| | target R | TA | sgRNA_A38994 |
| 175 | A_22974_Grik3 | ACCAATGCTGTCCAGTCCATTT | Amplification of Grik3 target site of |
| | target F | GC | sgRNA_A22974 |
| 176 | A_22974_Grik3 | TAGGCAAGCAGACAACACTAA | Amplification of Grik3 target site of |
| | target R | TGT | sgRNA_A22974 |
| 177 | A_47925_Serpinb9c | TCTTCTCAGGCTGAGAGTCAAT | Amplification of Serpinb9c target |
| | target-F | CC | site of sgRNA_A47925 |

### Example 2: Test of PB-CRISPR Vectors in Mouse iPS Cells

Mouse iPS cell line (iPS-ZX11-18-2) used was described previously (Wu, S., Wu, Y, Zhang, X. & Capecchi, M. R. Proc. Natl. Acad. Sci. 111, 10678-10683 (2014)). iPS cells were cultured in embryonic stem cell medium composed of DMEM (Gibco), 15% FBS (Gibco), 1×Penicillin and Streptomycin (Gibco) and 1000 U/mL LIF (Millipore). One million cells were electroporated with 1.5 μg pCRISPR-S10 that expresses Cas9 nuclease, 1.5 μg pCRISPR-sg6-Tet1/Tet2 and 1 μg pCAG-PBase. After electroporation, 1,000 cells were placed in a 10 cm dish. After 10 days, individual clones were picked for further culture and analysis. For PCR-RFLP assay, ˜500 bp DNA fragments around gRNA target sites were amplified using primers as previously published (Wang, H. Y. et al. Cell 153, 910-918 (2013)) from genomic DNA of iPS cells (Table 1), subjected to restriction endonuclease digestion and resolved on a 2% agarose gel. The result validated PB vector-mediated CRISPR mutagenesis by successfully targeting mouse Tet1 and Tet2 in cultured cells (FIG. 1b-d).

### Example 3: Library Construction

To construct the PB-CRISPR-M1 library, we synthesized oligos according to the genome-wide gRNA list (Shalem, O. et al. Science 343, 84-87 (2014)), amplified sgRNA with primer pair xc1732/xc1733, and cloned them into pCRISPR-sg6 at the BbsI site with the Gibson Assembly method (NEB). We amplified the sgRNA expression cassettes in the GeCKOv2 genome-scale mouse CRISPR/Cas9 knockout library (Sanjana, N. E., Shalem, O. & Zhang, F. Nat. Methods 11, 783-784 (2014)) including 130,209 synthesized sgRNA oligonucleotides targeting all mouse protein coding genes and miRNAs, and cloned into pCRISPR-sg6 to obtain the PB-CRISPR-M2 library (FIG. 2a).

To construct the PB-CRISPR-M2 library, we PCR amplified the U6-sgRNA cassettes from the GeCKOv2 mouse library (Sanjana, N. E., Shalem, O. & Zhang, F. Nat. Methods 11, 783-784 (2014)) and cloned them into pCRISPR-sg6.

For both PB-CRISPR-M1 library and PB-CRISPR-M2 library, 10 individual electroporations of 100 μL DH10B competent cells with 20 μL of ligation products were carried out. Bacterial cells were placed on one hundred 15 cm dishes to obtain about 10⁷ recombinants about 80-fold coverage of genome-wide gRNAs was obtained for PB-CRISPR M1 library, and about 10-fold coverage of genome-wide gRNAs was obtained for PB-CRISPR M2 library. Bacteria were harvested for maxi-preparation of PB-CRISPR libraries with the Endo-free Plasmid Maxi kit (Qiagen).

The integrity of this PB-CRISPR library was confirmed by deep sequencing, with 95% sgRNAs from the GeCKOv2 mouse library having representation in the PB-CRISPR-M2 library (FIG. 2b).

We also constructed a PB sgRNA library by cloning 130,209 synthesized sgRNA oligonucleotides into pCRISPR-sg6, resulting in the PB-CRISPR-M1 library. Due to simplicity of cloning, genome-wide PB-CRISPR libraries can be constructed rapidly, from synthesis of oligonucleotides to ready-for-use libraries in a week.

### Example 4: Deep Sequencing and Bioinformatics Analysis

Deep sequencing was used to profile the PB-CRISPR-M2 and GeCKOv2 libraries. After sequencing, we compared normalized read counts of gRNA between the two libraries and calculated Spearman correlation efficiency to measure their similarity (r²=0.83, P<0.001).

To identify sgRNA contents in tumors, —100 bp DNA fragments spanning the 20 nt gRNA region of PB library were PCR amplified from tumor genomic DNA or the library control. Sequencing libraries were constructed with these PCR products following standard protocols for the Illumina HiSeq2500. Individual libraries from different samples were barcoded and pooled. Sequences of ˜100 bp were demultiplexed from raw data and trimmed into 28 nt gRNA sequences containing sgRNA sequences, which were mapped against index libraries made from the GeCKOv2 library. Fully mapped reads were used to generate gRNA reads list.

To detect mutations in sgRNA target sites, we amplified ˜300 bp DNA including gRNA sequence in the center and performed NGS by Hiseq2500 following standard protocol. BWA aligner was used to map deep sequence data to the mouse genome (mm9) (Li, H. & Durbin, R. Bioinformatics 25, 1754-1760 (2009)). The bam files generated from BWA aligner were sorted and indexed by samtools (Li, H. et al. Bioinformatics 25, 2078-2079 (2009)). Mutation variants were called by VarScan.v2.3.9 (Koboldt, D. C. et al. Genome Res. 22, 568-576 (2012)).

### Example 5: Generation of Animal Model

All mouse experiments in this study were approved by the institutional animal care and use committees at China Agricultural University. CD-1 mice of 4 weeks old from Charles River were selected for hydrodynamic tail vein injection of PB-CRISPR library. It was shown that rapid injection of a large volume of DNA solution (˜10% of body weight) via mouse tail vein can achieve efficient gene transfer and expression in vivo, preferentially in the liver (Liu F, Song Y, & Liu D. Gene Ther 6(7), 1258-1266 (1999)). We followed a previously described injection protocol (Sanchez-Rivera, F. J. et al. Nature 516, 428-431 (2014)). The number of animals for screening and validation is derived from experience and confirmed with power analysis using data from prior, similar type studies (Chen, S. D. et al. Cell 160, 1246-1260 (2015); Sanchez-Rivera, F. J. et al. Nature 516, 428-431 (2014)). Mice were randomly allocated into different experimental groups. All mice injected were included for analysis. The investigators who assessing mice for tumorigenesis were blinded without knowing whether the animal was from control or experiment.

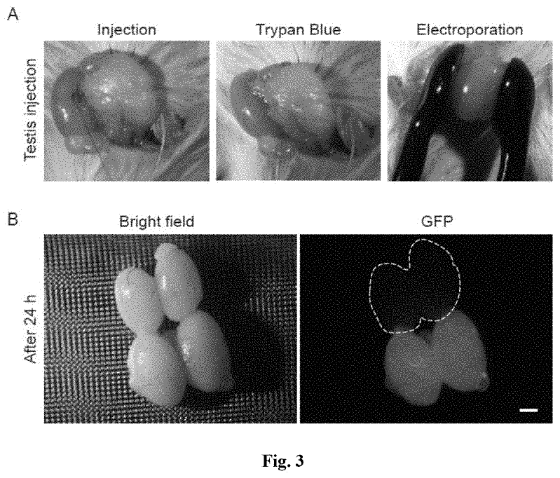

To evaluate the efficiency of delivery into mouse liver, we performed high pressure tail vein injection of the PB-CRISPR-M2 library, and pPB-IRES-EGFP, with or without PB transposase (PBase) overexpression plasmid pCAG-PBase, and analyzed liver samples at day 14 post injection (FIG. 2c). Strong and uniform GFP fluorescence across the entire liver could be detected when PBase was included (co-injected), while in contrast, the control group without PBase (n=3) had few GFP positive cells (FIG. 2c). Using deep sequencing to measure sgRNA representation in day 14 liver samples, on average 89.64±2.79% (n=3) of library sgRNAs were detected in each liver sample. Additionally, we confirmed that PB could be used for efficient transduction of other tissues, such as testis (FIG. 3). These results indicated that PB-mediated in vivo CRISPR delivery is very efficient.

To examine the in vivo library size after PB mediated delivery, 3 mice were injected with PB-CRISPR-M1 library, pPB-IRES-EGFP, pCAG-PBase at 8 μg each, and 3 Control mice (no pCAG-PBase) were injected with PB-CRISPR-M2 library and pPB-IRES-EGFP at 8 μg each. DNA was mixed in saline at a volume of 10% body weight. Each injection was finished within 10 seconds. Liver tissues (˜300 mg) were collected for genomic DNA extraction at day 14 post injection. sgRNAs were PCR amplified with primers listed in Table 1. The purified PCR products were used for NGS.

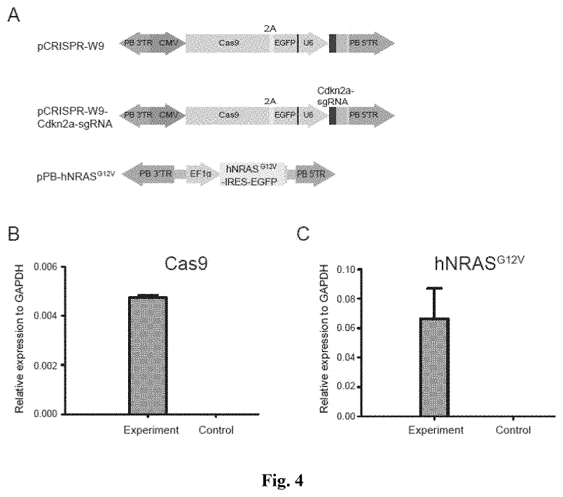

Since liver tumor screens typically require more than a year to obtain tumors (Bard-Chapeau, E. A. et al. Nat. Genet. 46, 24-32 (2014); Keng, V. W. et al. Nat. Biotechnol. 27, 264-274 (2009)), we aimed to find a faster scheme to demonstrate the feasibility of PB-CRISPR library screening in wild type mice. A recent CRISPR validation study showed that Cdkn2a sgRNA and Ras oncogene overexpression, with sgRNAs targeting 9 other TSGs delivered by SB transposon generated tumors, but only at 20-30 weeks after injection (Weber, J. et al. Proc. Natl. Acad. Sci. 112, 13982-13987 (2015)). We performed tail vein injections to test whether Cdkn2a-sgRNA/NRAS^{G12V} overexpression delivered by PB could be used as a sensitized genetic background. Total RNA was isolated from mouse liver using RNeasy Fibrous Tissue Mini Kit (Qiagen) following the manufacturer's protocol. RNA (2 μg) was reverse transcribed into cDNA using M-MLV reverse transcriptase (Promega). Quantitative RT-PCR was performed on LightCycler 480 (Roche) using LightCycler 480 SYBR Green I Master (Roche) following the program: pre-incubation (95° C., 10 sec), amplification (95° C., 10 sec; 60° C., 10 sec; 72° C., 10 sec) 30 cycles, melting curve (95° C., 5 sec; 65° C., 1 min), cooling (40° C., 10 sec). The primers used to detect the expression of Cas9 and hNRAS^{G12V} are displayed in Table 1. Gene expression was normalized to the GAPDH. We examined the 21 mice injected at day 61, and no tumors were detected (Table 2), while Cas9 and NRAS^{G12V} expression could be detected by quantitative real-time RT-PCR (qRT-PCR) in liver samples (FIG. 4) from these mice. This result indicated that the sensitized background of Cdkn2a sgRNA/NRAS^{G12V} could be ideal for rapid screening within 2 months, as an additional trigger from the PB-CRISPR library could accelerate tumor formation.

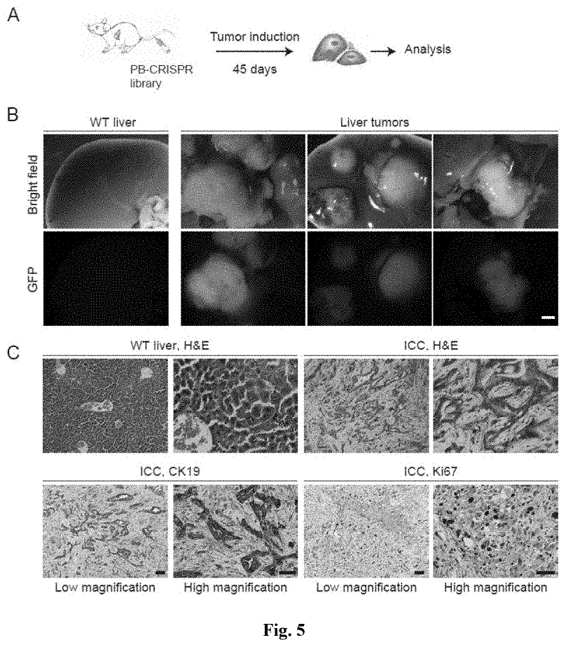

We next conducted a genome-wide screen for liver tumorigenesis through injection with pCRISPR-W9-Cdkn2a-sgRNA, pPB-hNRAS^{G12V}, and the PB-CRISPR-M2 library, along with pCAG-PBase (FIG. 5a and Table 2) into 27 mice. pCRISPR-W9-Cdkn2a-sgRNA expresses Cas9 and EGFP linked by 2A self-cleavage peptide and Cdkn2a sgRNA. pPB-hNRAS^{G12V} is a PB plasmid expressing NRAS with G12V dominant mutation and IRES-EGFP. All 27 mice injected were examined at 45 days post injection when the first mouse in this group died with a tumor. Liver tumors developed in 9 out of 27 mice, with each mouse containing 1-9 tumors, but no tumors were detected outside the liver. Tumors were readily detected due to their large size (˜5 mm-20 mm) and strong GFP fluorescence (FIG. 5b).

TABLE 2
PB-CRISPR library screening for tumorigenesis in mouse livers.

| | pCRISPR-W9- | pPB- | PB-CRISPR- | pCAG- | Tumorigenesis | |
|---|---|---|---|---|---|---|
| | Cdkn2a-sgRNA | hNRAS^{G12V} | M2 library | PBase | efficiency (%) | |
| Control | 12 μg | 12 μg | — | 8 μg | 0/21 | (♂, 0%) |
| Screen | 8 μg | 8 μg | 8 μg | 8 μg | 9/27 | (♂, 33.3%) |

Note:
In addition to the 27 male mice in the screening group, we also performed screening with 20 female mice that were not included in the table. No tumor induction was observed in the 20 female mice at day 61. It is known that male mice are many fold more likely to develop liver tumors than female mice (Naugler, W.E. et al. Science 317, 121-124 (2007)).

### Example 6: Hydrodynamic Tail Vein Injection of PB-CRISPR Library and Detection of Tumors

To examine the in vivo library size after PB mediated delivery, 3 mice were injected with PB-CRISPR-M1 library, pPB-IRES-EGFP, pCAG-PBase at 8 μg each, and 3 Control mice (no pCAG-PBase) were injected with PB-CRISPR-M2 library and pPB-IRES-EGFP at 8 μg each. DNA was mixed in saline at a volume of 10% body weight. Each injection was finished within 10 seconds. Liver tissues (˜300 mg) were collected for genomic DNA extraction at day 14 post injection. sgRNAs were PCR amplified with primers listed in Table 1. The purified PCR products were used for NGS. Deep sequencing was used to profile the PB-CRISPR-M2 and GeCKOv2 libraries. After sequencing, we compared normalized read counts of gRNA between the two libraries and calculated Spearman correlation efficiency to measure their similarity (r²=0.83, P<0.001).

For in vivo screening, each mouse was injected with pCRISPR-W9-Cdkn2a-sgRNA, pPB-hNRAS^{G12V}, PB-CRISPR-M2 library and pCAG-PBase at 8 μg each in saline at a volume of 10% body weight. Control groups were injected with plasmids according to Table 2.

For validation experiments, each mouse was injected with corresponding PB-sgRNA, pCRISPR-W9-Cdkn2a-sgRNA (or pCRISPR-W9), pPB-hNRAS^{G12V}, and pCAG-PBase at 8 μg each in saline at a volume of 10% body weight. On the day the first mouse in a group died, all mice in the same group were examined. If no mice died in a validation group, all mice were examined at day 45 post injection. For the control group, mice were examined at day 61 post injection.

Tumors were fixed in 4% formalin in PBS at 4° C. overnight, paraffin embedded, sectioned at 5 μm and stained with hematoxylin and eosin (H&E) for pathology. The following antibodies were used for immunostaining: Anti-Actin, a-Smooth Muscle antibody, Mouse monoclonal clone 1A4 (Sigma, A5228); Monoclonal anti-vimentin clone LN-6 (Sigma, V2258); Anti-Collagen Type IV Antibody (EMD Millipore Corporation, AB8201); Anti-alpha 1 Fetoprotein antibody (Abcam, ab46799); Purified Mouse Anti-Ki-67 (BD, 550609); Anti-Cytokeratin AE1/AE3 antibody (Abcam, ab115963). The pathologists reading the slides were blinded.

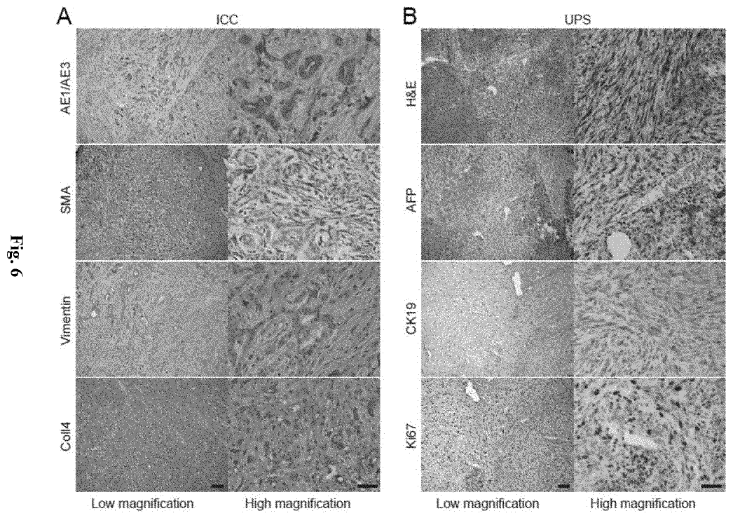

Histological analysis by hematoxylin and eosin (H&E) staining and immunohistochemistry showed that most tumors analyzed were intrahepaticcholangiocarcinoma (ICC) (FIG. 5c and FIG. 6), consistent with previous observations that most tumors induced in mouse liver tumor models are ICCs (Xue, W. et al. Nature 514, 380-384 (2014); Weber, J. et al. Proc. Natl. Acad. Sci. 112, 13982-13987 (2015)). Additionally, two tumors appeared to be undifferentiated pleomorphic sarcoma (UPS) (FIG. 6), which has not been reported in mouse liver cancer models, but suggests that transfection of non-hepatocytes such as stromal cells might have also contributed to liver tumors. The results of rapid tumor formation demonstrated that PB-mediated CRISPR library delivery is practical for in vivo screening in mice.

### Example 7: Sequencing and Identification of sgRNA Contents in Tumor

To identify sgRNA contents in tumors, —100 bp DNA fragments spanning the 20 nt gRNA region of PB library were PCR amplified from tumor genomic DNA or the library control. Sequencing libraries were constructed with these PCR products following standard protocols for the Illumina HiSeq2500. Individual libraries from different samples were barcoded and pooled. Sequences of ˜100 bp were demultiplexed from raw data and trimmed into 28 nt gRNA sequences containing sgRNA sequences, which were mapped against index libraries made from the GeCKOv2 library. Fully mapped reads were used to generate gRNA reads list.

To detect mutations in sgRNA target sites, we amplified ˜300 bp DNA including gRNA sequence in the center and performed NGS by Hiseq2500 following standard protocol. BWA aligner was used to map deep sequence data to the mouse genome (mm9) (Li H & Durbin R. Bioinformatics 25(14):1754-1760.(2009)). The bam files generated from BWA aligner were sorted and indexed by samtools (Li H, et al. Bioinformatics 25(16):2078-2079 (2009)). Mutation variants were called by VarScan.v2.3.9 (Koboldt D C, et al. Genome research 22(3):568-576 (2012)).

To identify sgRNAs that had inserted into the tumor genome, we selected 18 tumors for further analysis. We used PCR to amplify sgRNAs from each tumor for next generation sequencing (NGS). We generated a list of 1149 TSG orthologs in mouse genome using human TSG as comparative information (http://bioinfo.mc.vanderbilt.edu/TSGene) (Zhao M, Sun J, & Zhao Z Nucleic Acids Res 41 (Database issue):D970-976. (2013)). In the PB-CRISPR libraries, there were 6650 sgRNAs targeting all these mouse TSG orthologs. Out of 271 sgRNAs identified in 18 tumors, 26 sgRNAs targeting 21 mouse TSG orthologs were found to be significantly enriched (P<0.01) by two-sided Fisher's Exact test.

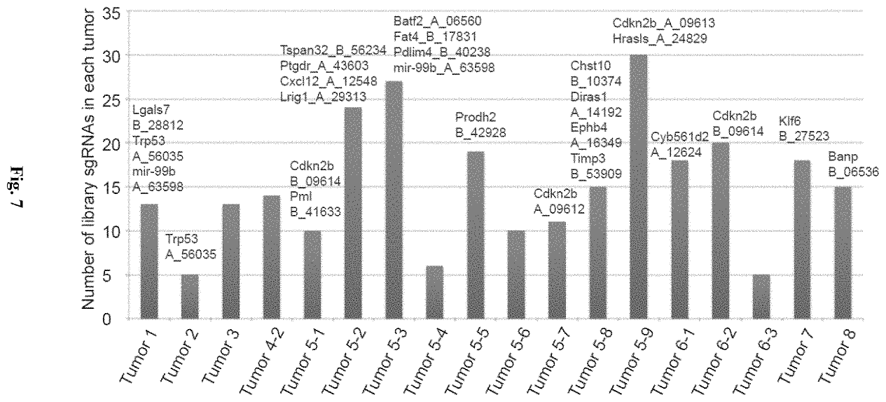

A total of 271 library sgRNAs was identified, with each tumor containing 15.06±7.64 sgRNAs (Table 3). The differences in counts for sgRNAs within a tumor suggest that some tumors may have a multiclonal origin. Also, the differences in sgRNA content for tumors isolated from one mouse (i.e., Tumor 5-1 to Tumor 5-8) showed they were clonally unrelated. Among the 271 sgRNAs, the prominent TSG Trp53 was targeted twice, and Cdkn2b, a TSG not previously implicated in mouse liver cancers (Krimpenfort P, et al. Nature 448(7156):943-946 (2007)), was targeted in 4 tumors by 3 distinct sgRNAs (Table 4). In total, 26 of the 271 sgRNAs were targeting 21 mouse TSG orthologs. Analysis by Fisher's exact test found these sgRNAs for TSGs were significantly enriched (P<0.01, FIG. 7, Table 3) (Zhao M, Sun J, & Zhao Z. Nucleic Acids Res 41(Database issue):D970-976. (2013)).

TABLE 3
Sequencing read counts of sgRNA contents in tumors and CRISPR libraries.

| Tumor 1 | reads | Tumor 2 | reads |
|---|---|---|---|
| Non_Lib Cdkn2a sgRNA | 178117 | Non_Lib Cdkn2a sgRNA | 683716 |
| LibA_54001_Tle4 | 79496 | LibA_24528_Hmox2 | 195420 |
| LibA_36261_Olfrl311 | 75666 | LibB_30072_Magel2 | 166531 |
| LibB_28812_Lgals7 | 74390 | LibA_38994_Osbpl11 | 159980 |
| LibA_28412_Lamc2 | 73428 | LibA_56035_Trp53 | 30905 |
| LibA_56035_Trp53 | 72553 | LibA_22974_Grik3 | 11746 |
| LibB_27405_Kif5a | 41358 | | |
| LibB_51283_Srms | 39028 | | |
| LibA_07138_Brap | 38781 | | |
| LibA_26940_Kcnh1 | 37981 | | |
| LibB_25175_Hyal5 | 35944 | | |
| LibB_21517_Gm4981 | 22964 | | |
| LibA_02125_AK129341 | 17831 | | |
| LibA_63598_mmu-mir-99b | 15705 | | |

| Tumor 3 | reads | Tumor 4-2 | reads |
|---|---|---|---|
| Non_Lib Cdkn2a sgRNA | 349083 | Non_Lib Cdkn2a sgRNA | 346022 |
| LibA_08684_Ccdc87 | 119186 | LibA_29995_Mael | 203340 |
| LibB_34017_Ninj2 | 113996 | LibB_49633_Slc6a14 | 187983 |
| LibA_30543_Mbd6 | 109867 | LibB_47599_Sel1l2 | 173556 |
| LibB_01105_4430402I18Rik | 99285 | LibA_44928_Rcbtb2 | 166965 |
| LibB_53696_Thbs2 | 98008 | LibB_15909_Elane | 115690 |
| LibA_28398_Lama5 | 97720 | LibB_01812_9130204L05Rik | 57159 |
| LibB_19120_Fxyd4 | 95392 | LibA_60072_Zbtb37 | 56058 |
| LibA_36051_Olfr1239 | 94355 | LibA_28484_Larp6 | 48306 |
| LibA_45045_Reep5 | 90491 | | |
| LibB_10774_Clec5a | 87335 | | |
| LibA_63693_mmu-mir-6970 | 86327 | | |
| LibB_21757_Gm5941 | 53975 | | |
| LibB_00272_1700010D01Rik | 53412 | | |

| Tumor 5-1 | reads | Tumor 5-2 | reads |
|---|---|---|---|
| Non_Lib Cdkn2a sgRNA | 519570 | Non_Lib Cdkn2a sgRNA | 159353 |
| LibA_36065_Olfr1243 | 113721 | LibA_45974_Rnf41 | 66438 |
| LibA_60658_Zfp35 | 60292 | LibB_00914_2610008E11Rik | 58889 |
| LibB_17813_Fastkd5 | 59459 | LibB_40856_Piga | 57336 |
| LibB_09614_Cdkn2b | 57848 | LibA_23193_Gstm6 | 55389 |
| LibB_41633_Pml | 57456 | LibB_07516_C2cd5 | 35674 |
| LibA_53331_Tecr | 56282 | LibB_15677_Egln2 | 29171 |
| LibA_35899_Olfr1181 | 52368 | LibB_56234_Tspan32 | 28000 |
| LibA_55795_Trim36 | 49251 | LibA_54542_Tmem204 | 27549 |
| LibB_22324_Golga7 | 23205 | LibA_48907_Slc22a13 | 21054 |
| | | LibB_35753_Olfr1124 | 19699 |
| | | LibB_41670_Pnldc1 | 19393 |
| | | LibB_51847_Stpg1 | 19052 |
| | | LibA_24448_Hmbs | 18900 |
| | | LibB_05253_Arpc2 | 18719 |
| | | LibA_43603_Ptgdr | 18545 |
| | | LibB_25109_Htr2c | 18487 |
| | | LibB_16894_F11r | 18259 |
| | | LibA_12548_Cxcl12 | 17780 |
| | | LibB_45181_Rffl | 17726 |
| | | LibA_41775_Podnl1 | 17588 |
| | | LibB_11689_Cplx2 | 16962 |
| | | LibB_47900_Serpinb7 | 16538 |
| | | LibA_60429_Zfp119b | 16101 |
| | | LibA_33403_Nckap1 | 16081 |
| | | LibA_29313_Lrig1 | 15791 |
| | | LibA_65968_mmu-mir-190a | 15125 |

| Tumor 5-3 | reads | Tumor 5-4 | reads |
|---|---|---|---|
| Non_Lib Cdkn2a sgRNA | 437562 | Non_Lib Cdkn2a sgRNA | 420576 |
| LibB_39752_Pcdha7 | 74428 | LibB_39516_Parm1 | 149148 |
| LibA_25389_Ifitm10 | 59669 | LibA_05076_Arid3a | 51927 |
| LibA_61878_mmu-mir-6899 | 55426 | LibA_14919_Drosha | 41430 |
| LibA_52111_Sun5 | 53318 | LibA_01192_4930402H24Rik | 39138 |
| LibB_08039_Card10 | 46197 | LibB_54964_Tmprss11g | 38160 |
| LibB_40945_Pik3ca | 45558 | LibA_64958_mmu-mir-7024 | 26097 |
| LibA_34695_Nr3c2 | 43931 | | |
| LibB_26738_Kalrn | 41919 | | |
| LibA_06560_Batf2 | 41821 | | |
| LibB_49468_Slc3a2 | 41319 | | |
| LibB_17831_Fat4 | 40673 | | |
| LibB_01544_4933406M09Rik | 39333 | | |
| LibB_56849_Ubash3b | 37997 | | |
| LibA_24184_Hip1 | 37410 | | |
| LibB_15961_Ell | 36507 | | |
| LibB_02143_AU022252 | 36309 | | |
| LibB_09073_Cd226 | 36151 | | |
| LibA_66012_mmu-mir-7088 | 35294 | | |
| LibA_01258_4930444P10Rik | 35078 | | |
| LibB_38229_Olfr749 | 34955 | | |
| LibA_30114_Mal2 | 34779 | | |
| LibB_40238_Pdlim4 | 34671 | | |
| LibB_34799_Nrtn | 34388 | | |
| LibB_23705_Hars2 | 32513 | | |
| LibB_22105_Gm9 | 30709 | | |
| LibA_63598_mmu-mir-99b | 25488 | | |
| LibB_20082_Gli2 | 23750 | | |
| LibB_44971_Rdh12 | 20585 | | |
| LibB_00946_2610507B11Rik | 18384 | | |

| Tumor 5_5 | reads | Tumor 5-6 | reads |
|---|---|---|---|
| Non_Lib Cdkn2a sgRNA | 344749 | Non_Lib Cdkn2a sgRNA | 256253 |
| LibA_60936_Zfp58 | 81510 | LibB_55404_Tpd52 | 172586 |
| LibB_52439_Syvn1 | 55188 | LibB_41976_Pon2 | 168052 |
| LibA_19117_Fxyd1 | 55131 | LibA_53332_Tecr | 76909 |
| LibB_51502_Ssxb2 | 52977 | LibA_47925_Serpinb9c | 60232 |
| LibB_58325_Vmn1r32 | 51702 | LibB_44752_Rbm15 | 57035 |
| LibB_23010_Grk1 | 49789 | LibA_12850_Cyp2g1 | 52435 |
| LibA_29632_Lsg1 | 44093 | LibA_39356_Paip2b | 42966 |
| LibB_56483_Ttll11 | 29847 | LibB_23557_H2-Q2 | 28699 |
| LibA_26034_Inhbb | 29357 | LibB_16359_Epm2aip1 | 18351 |
| LibB_42928_Prodh2 | 29097 | | |
| LibB_49525_Slc46a3 | 28516 | | |
| LibB_52503_Tab3 | 28091 | | |
| LibB_38405_Olfr825 | 27805 | | |
| LibB_10173_Chd8 | 26702 | | |
| LibB_35565_Olfr1040 | 26572 | | |
| LibA_18146_Fcgrt | 25848 | | |
| LibB_55502_Tra2a | 25796 | | |
| LibA_17421_Fam207a | 25523 | | |
| LibB_44549_Rarres2 | 24875 | | |
| LibB_13009_Cypt3 | 20325 | | |

| Tumor 5-7, Xcl803_804 | reads | Tumor 5-8 | reads |
|---|---|---|---|
| Non_Lib Cdkn2a sgRNA | 557730 | Non_Lib Cdkn2a sgRNA | 720448 |
| LibB_19898_Gif | 394377 | LibB_49455_Slc39a7 | 898551 |
| LibB_31702_Morn1 | 380768 | LibB_53909_Timp3 | 680480 |
| LibA_09612_Cdkn2b | 379473 | LibB_40853_Pifo | 631964 |
| LibA_19126_Fxyd4 | 360222 | LibB_10374_Chst10 | 549417 |
| LibB_44494_Rap1gap2 | 192583 | LibB_60451_Zfp169 | 438788 |
| LibB_57494_Usp14 | 160978 | LibA_19159_Fzd3 | 413102 |
| LibA_33521_Ndufa11 | 156299 | LibA_16349_Ephb4 | 346490 |
| LibA_46638_Rspry1 | 154153 | LibA_35927_Olfr1195 | 335671 |
| LibA_57601_Usp38 | 153594 | LibB_32542_Mtrf1 | 296276 |
| LibB_06455_Baat | 151006 | LibB_23312_Gtpbp6 | 278908 |
| LibA_18990_Fsd1l | 150912 | LibB_22204_Gnb1l | 241183 |
| LibB_54358_Tmem151a | 103753 | LibA_14192_Diras1 | 183056 |
| LibA_58144_Vmn1r187 | 87832 | LibA_04825_Arcn1 | 156153 |
| LibA_58447_Vmn1r63 | 86398 | LibA_36096_Olfr1253 | 138404 |
| | | LibB_16547_Ero1l | 132065 |
| | | LibA_36078_Olfr1248 | 62184 |

| Tumor 5-9 | | reads | Tumor 6-1 | reads |
|---|---|---|---|---|
| Non_Lib Cdkn2a sgRNA | | 264447 | Non_Lib Cdkn2a sgRNA | 368121 |
| LibA_57366_Uox | | 73405 | LibA_17464_Fam216a | 113770 |
| LibB_26396_Ism1 | | 73241 | LibA_45041_Reep3 | 80199 |
| LibB_29348_Lrp4 | | 72812 | LibB_34463_Noxa1 | 79634 |
| LibB_34951_Ntsr2 | | 68839 | LibA_51486_Sstr4 | 72958 |
| LibA_10483_Cirbp | | 68255 | LibB_59988_Zbed4 | 66525 |
| LibB_53715_Them6 | | 66741 | LibA_21983_Gm8267 | 58878 |
| LibB_35552_Olfr1036 | | 65267 | LibA_33671_Necap2 | 55764 |
| LibB_18821_Foxn4 | | 65121 | LibA_62608_mmu-mir-7675 | 54543 |
| LibA_59865_Ybey | | 64282 | LibB_03015_Adamdec1 | 46663 |
| LibB_18541_Flg2 | | 39408 | LibA_41372_Plcxd3 | 45851 |
| LibA_65134_mmu-mir-7038 | | 37737 | LibB_19470_Gart | 41490 |
| LibA_08613_Ccdc66 | | 36439 | LibB_27554_Klhdc8a | 37507 |
| LibA_34733_Nrbp2 | | 36428 | LibB_14309_Dlx5 | 35096 |
| LibB_14892_Drd2 | | 35058 | LibA_08107_Casp1 | 34261 |
| LibB_01710_6330403K07Rik | | 35054 | LibA_02512_Acaa1a | 33977 |
| LibA_65124_mmu-mir-106b | | 35046 | LibB_05210_Armc7 | 32057 |
| LibA_09613_Cdkn2b | | 34893 | LibA_12624_Cyb561d2 | 32043 |
| LibA_55631_Trappc6b | | 34466 | LibB_57704_Uvssa | 31753 |
| LibA_31165_Mfsd3 | | 34265 | LibA_53129_Tcf23 | 28339 |
| LibB_14205_Disp1 | | 34190 | | |
| LibB_23798_Hbs1l | | 33917 | | |
| LibA_48501_Sike1 | | 33618 | | |
| LibA_48721_Slc11a2 | | 33487 | | |
| LibA_32679_Mup4 | | 33401 | | |
| LibA_30517_Mb21d2 | | 33289 | | |
| LibB_44971_Rdh12 | | 33019 | | |
| LibA_24829_Hrasls | | 32972 | | |
| LibA_38972_Orm3 | | 31859 | | |
| LibB_52238_Sybu | | 30887 | | |
| | Tumor 6-2 | reads | Tumor 6-3 | reads |
| | Non_Lib Cdkn2a sgRNA | 294384 | Non_Lib Cdkn2a sgRNA | 315812 |
| | LibA_18221_Fer1l5 | 74571 | LibB_01749_7420426K07Rik | 139127 |
| | LibB_41378_Pld5 | 56460 | LibB_02162_AW209491 | 69609 |
| | LibA_38419_Olfr827 | 52438 | LibA_10308_Chrd | 69515 |
| | LibA_45229_Rfx2 | 31383 | LibB_07911_Cap1 | 65520 |
| | LibB_52314_Syne3 | 30058 | LibB_50056_Smg5 | 65089 |
| | LibB_23042_Grm7 | 29876 | | |
| | LibA_30721_Mcts2 | 28533 | | |
| | LibB_09614_Cdkn2b | 28296 | | |
| | LibB_46980_Sap25 | 27609 | | |
| | LibA_27300_Khdrbs1 | 27222 | | |
| | LibB_08605_Ccdc64b | 26685 | | |
| | LibA_06409_BC061194 | 26671 | | |
| | LibB_04082_Anapc11 | 26327 | | |
| | LibA_06437_BC100451 | 26274 | | |
| | LibA_19051_Fubp3 | 26248 | | |
| | LibB_36191_Olfr1289 | 26175 | | |
| | LibB_10820_Clk2 | 26098 | | |
| | LibB_40967_Pik3r3 | 25565 | | |
| | LibB_54803_Tmem5 | 25068 | | |
| | LibA_49111_Slc25a43 | 24707 | | |

| Tumor 7 | reads | Tumor 8 | reads |
|---|---|---|---|
| Non_Lib Cdkn2a sgRNA | 134486 | Non_Lib Cdkn2a sgRNA | 276203 |
| LibB_36840_Olfr180 | 70029 | LibA_43296_Psap | 107483 |
| LibA_50088_Smgc | 53014 | LibA_06193_B4galt1 | 87066 |
| LibB_39421_Panx1 | 52724 | LibB_06536_Banp | 56283 |
| LibA_55968_Trmt1 | 51603 | LibA_18923_Frmd3 | 30606 |
| LibA_58478_Vmn1r72 | 47331 | LibA_64315_mmu-mir-6998 | 30573 |
| LibA_08492_Ccdc17 | 27654 | LibB_59778_Xpo7 | 29207 |
| LibB_59038_Vsig10 | 27600 | LibB_59489_Wfs1 | 28632 |
| LibA_10807_Clip1 | 24935 | LibA_45157_Rev1 | 27488 |
| LibB_49250_Slc30a6 | 22867 | LibA_02488_Ablim3 | 26908 |
| LibA_10210_Chid1 | 22274 | LibB_30520_Mbd3l2 | 26495 |
| LibA_54751_Tmem39b | 22145 | LibB_04104_Anapc7 | 25382 |
| LibB_24840_Hrh2 | 20682 | LibA_46942_Samd14 | 25292 |
| LibB_27523_Klf6 | 20677 | LibB_13899_Dennd3 | 16711 |
| LibA_29077_Lmbr1l | 20198 | LibB_05135_Arl2bp | 14325 |
| LibB_32862_Myl12a | 20029 | | |
| LibA_00425_1700025G04Rik | 17153 | | |

127417 genes in PB-CRISPR-M2

TABLE 4
Genes that have been targeted twice or more.

| Gene | sgRNA | sgRNA | sgRNA | sgRNA |
|---|---|---|---|---|
| Cdkn2b | B_09614_Cdkn2b | A_09612_Cdkn2b | A_09613_Cdkn2b | B_09614_Cdkn2b |
| | Tumor 5-1 | Tumor 5-7 | Tumor 5-9 | Tumor 6-2 |
| Fxyd4 | B_19120_Fxyd4 | A_19126_Fxyd4 | | |
| | Tumor 3 | Tumor 5-7 | | |
| mir-99b | A_63598_mmu-mir-99b | A_63598_mmu-mir-99b | | |
| | Tumor 1 | Tumor 5-3 | | |
| Rdh12 | B_44971_Rdh12 | B_44971_Rdh12 | | |
| | Tumor 5-3 | Tumor 5-9 | | |
| Tecr | A_53331_Tecr | A_53332_Tecr | | |
| | Tumor 5-1 | Tumor 5-6 | | |
| Trp53 | A_56035_Trp53 | A_56035_Trp53 | | |
| | Tumor 1 | Tumor 2 | | |

Since each tumor in our screen contained multiple copy sgRNA insertions, we tested whether large deletions and translocations resulted from targeting by two sgRNAs could have made some contribution to tumorigenesis, as suggested by previous reports (Maddalo D, et al. Nature 516(7531):423-+(2014); Blasco R B, et al. Cell reports 9(4):1219-1227 (2014)) To survey this possibility, we chose 7 tumors: Tumor 1, 2, 3, 4-2, 5-4, 5-6, and 5-7, and performed PCR reactions with all possible combinations of primers (Table 1). However, no translocations and large deletions in 7 tumors were detected. Previous reports suggested that insertional mutagenesis by multiple transposon insertions could contribute to tumorigenesis (Bard-Chapeau E A, et al. Nature genetics 46(1):24-32 (2014); Carlson C M, et al., Proceedings of the National Academy of Sciences of the United States of America 102(47):17059-17064 (2005); Keng V W, et al. 27(3):264-274 (2009); Dupuy A J, et al. Nature 436(7048):221-226 (2005)). However, considering that the control group was injected with the same amount of PB vectors (Table 2) but did not develop any tumor, tumors obtained from the screen should be largely attributed to library-mediated CRISPR mutagenesis. Taken together, these analyses suggest that identified TSGs could be the main reason for the increased tumorigenesis in the screen.

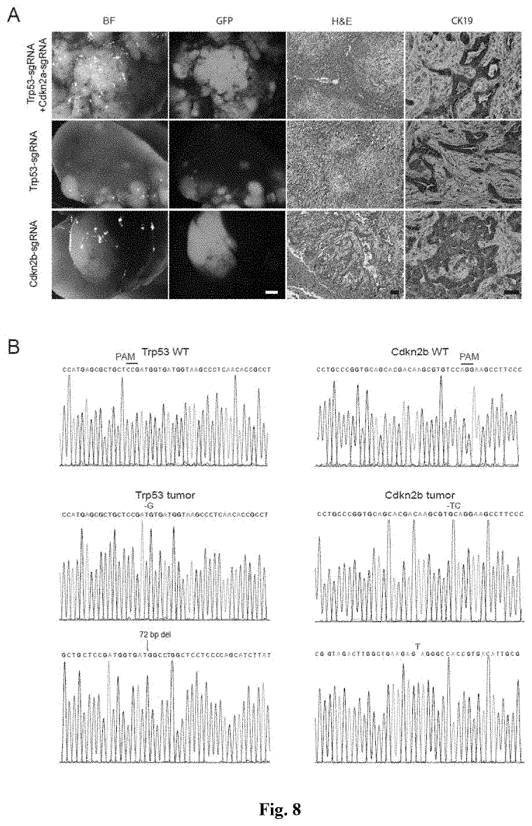

We next tested sgRNA of the prominent Trp53 to verify whether it would contribute to accelerated tumor formation in our PB delivery system. In the Trp53 group with Cdkn2a-sgRNA, all mice were examined at day 21 post injection, when the first mouse in this group died of tumors (FIG. 8a and Table 5). Strikingly, 10 out of 11 mice injected developed liver tumors, with tumor numbers ranging from a few to >100. To validate Trp53-sgRNA more definitively, we performed injections of Trp53-sgRNA without Cdkn2a-sgRNA. All mice were examined at day 28 post injection, and 8 out of 11 mice developed liver tumors (FIG. 8a and Table 5).

TABLE 5
Validated TSGs in liver tumorigenesis.

| | | pCRISPR-W9- | pPB- | pCAG- | Tumorigenesis | |
|---|---|---|---|---|---|---|
| sgRNA_target gene | pCRISPR-W9 | Cdkn2a-sgRNA | hNRAS^{G12V} | PBase | efficiency (%) | |
| A_56035_Trp53 | − | + | + | + | 10/11 | (♂, 90.9%) |
| A_56035_Trp53 | + | − | + | + | 8/11 | (♂, 72.7%) |
| B_09614_Cdkn2b | − | + | + | + | 11/11 | (♂, 100%) |
| B_09614_Cdkn2b | + | − | + | + | 4/11 | (♂, 36.4%) |
| Control group | − | + | + | + | 0/20 | (♂, 0%) |

We further conducted validation experiments for sgRNA of Cdkn2b, whose tumor suppressor role has not been previously implicated in mouse liver cancers. In the Cdkn2b-sgRNA group with Cdkn2a-sgRNA, at 21 days post injection, 11 out of 11 mice developed liver tumors (Table 5), with tumor numbers in each mouse >100, a big increase compared to screening experiments. In the Cdkn2b-sgRNA group, at 45 days post injection, 4 out of 11 mice developed liver tumors (FIG. 8a and Table 5), with tumor numbers ranging from 1-3, indicating that Cdkn2b alone could be a potent TSG in liver tumorigenesis. Additionally, mutations in the target regions of Trp53 and Cdkn2b tumors were confirmed (FIG. 8b). Together, these results demonstrate the rapidity and efficiency of PB-CRISPR for in vivo screening, and proved that sgRNAs for known and novel TSGs in the screen could be readily recovered.

### Example 8: Comparison of PB-CRISPR Library with Previous Methods

Previously, genome-wide gRNA lentiviral library was used to screen for 6-thioguanine resistant clones (Koike-Yusa et al., 2014). ES cells were first infected with lentiviral library followed by FACS sorting and expansion. 10×10⁶ mutant ESCs were treated with 6TG (2 M) for 5 d, and further cultured for an additional 5 d, thus obtaining 6TG resistant clones.

In comparison, we performed a PB-CRISPR library screening. ES cells were first electroporated with PB-CRISPR library. These cells were then directly used for 6TG selection, and clones were obtained 2 times faster than previous methods.

In the present invention, PB-CRISPR method has provided an efficient approach to conduct direct in vivo CRISPR library screening, as well as rapid in vivo validation of cancer genes. Compared to previous indirect in vivo screening by transplanting cultured cells (Chen S D, et al. (2015) Genome-wide CRISPR Screen in a Mouse Model of Tumor Growth and Metastasis. Cell 160(6):1246-1260.), the method of the present invention is much simpler and more likely to reveal relevant TSGs by recapitulating the complexity of the in vivo environment. In this proof-of-principle study, the application focused on a fast screening scheme, which by design is more likely to recover mutational events for early tumor occurrence, but with longer incubation time or other genetic backgrounds tumors with different mutational profiles should develop in the screening. With the increase of sample numbers, it may be possible to obtain a more complete list of TSGs involved in liver cancer development.

In the present invention, PB-CRISPR method has some advantages, for example, copy number of PB-CRISPR library can be flexibly controlled, and the screening of PB-CRISPR library can be directly in vivo.

Furthermore, this speed of tumor screening and validation in the invention is unprecedented, e.g., in the validation experiments for Cdkn2b sgRNA, numerous tumors developed within liver in less than 3 weeks. In contrast, similar previous in vivo tumor modeling using CRISPR and SB transposon or pX330 plasmid required a much longer time for tumor formation (Xue W, et al. (2014) CRISPR-mediated direct mutation of cancer genes in the mouse liver. Nature 514(7522):380-384; Weber J, et al. (2015) CRISPR/Cas9 somatic multiplex-mutagenesis for high-throughput functional cancer genomics in mice. Proceedings of the National Academy of Sciences of the United States of America 112(45):13982-13987.). One possible explanation is that PB mediates very efficient stable transposition in most hydrodynamically injected liver cells (FIG. 2). In the future, combined with other innovative delivery methods, such as nanoparticles and electroporation (Zuckermann M, et al. (2015) Somatic CRISPR/Cas9-mediated tumour suppressor disruption enables versatile brain tumour modelling. Nature Communications 6:9; Platt R J, et al. (2014) CRISPR-Cas9 Knockin Mice for Genome Editing and Cancer Modeling. Cell 159(2):440-455.), the extreme simplicity of PB-CRISPR libraries should greatly enhance the already powerful CRISPR weaponry.

### SEQUENCE LISTING

The sequence listing submitted herewith in the ASCII text file entitled “A002US1_ST25 Sequence Listing,” created Sep. 16, 2019, with a file size of 33.897 kilobytes, is incorporated herein by reference in its entirety.

1. A genome wide library comprising:
a plurality of PB-mediated CRISPR system polynucleotide, comprising minimal guide RNAs flanked by minimal piggyBac inverted repeat elements, and said guide sequences are capable of targeting a plurality of target sequences of interest in a plurality of genomic loci in a population of eukaryotic cells, tissues, or organisms.

2. The library of claim 1, wherein the population of eukaryotic cells is a population of mammalian cells such as mouse cells or human cells.

3. The library of claim 1, wherein the population of eukaryotic cells is a population of any kind of cells such as fibroblast.

4. The library of claim 1, wherein the population of tissues is a population of any kind of the non-reproductive tissues such as liver or lungs.

5. The library of claim 1, wherein the population of organisms is a population of mouse.

6. The library of claim 1, wherein the target sequence in the genomic locus is a coding sequence.

7. The library of claim 1, wherein gene function of said target sequence is altered by said targeting.

8. The library of claim 1, wherein said targeting results in a knockout of gene function.

9. The library of claim 1, wherein the targeting is of the entire genome.

10. The library of claim 8, wherein the knockout of gene function is achieved in a plurality of unique genes which function in mediating tumorigenesis, anti-aging, and longevity.

11. The library of claim 10, wherein said unique gene is tumor suppressor gene.

12. A method of in vivo genome-scale screening comprising:
(a) introducing into a mammal containing and expressing a RNA polynucleotide having a target sequence,
(b) encoding at least one gene product of a PB-mediated CRISPR system comprising one or more vectors comprising:
(i) a first polynucleotide encoding a Cas9 protein, or a variant thereof or a fusion protein therewith,
(ii) a second polynucleotide encoding a PB transposase, or a variant thereof or a fusion protein therewith,
(iii) a third polynucleotide library of claims 1-11,
wherein components (i), (ii), and (iii) are located on same or different vectors of the system,
whereby PB transposase introduce guide RNA into genomes, the guide RNA targets the target sequence an Cas9 protein generates at least one site specific break is repaired through a cellular repair mechanism,
(c) amplifying and sequencing the genomic DNA from said mammal.

13. The method of claim 12, wherein gene function of said gene product is altered by said system.

14. The method of claim 12, wherein said system results in a knockout of gene function.

15. The method of claim 14, wherein the knockout of gene function is achieved in a plurality of unique genes which function in mediating tumorigenesis, anti-aging, and longevity.

16. The method of claim 12, wherein said mammal in step (a) expresses at least one oncogene or knockouts at least one tumor suppresser gene to generate a sensitized background for screening without tumor formation.

17. The method of claim 16, wherein said oncogene is NRAS with dominant G12V mutation.

18. The method of claim 16, wherein said tumor suppresser gene is selected from the group consists of Cdkn2b, Trp53, Klf6, miR-99b, Clec5a, Selll2, Lgals7, Pml, Ptgdr, Tspan32, Fat4, Pik3ca, Pdlim4, Cxcl12, Lrig1, Batf2, Pmdh2, Chst10, Diras1, Ephb4, Timp3, Hrasls, Banp, and Cyb56Id2.

19. The method of claim 12, wherein said mammal is mouse.

20. The method of claim 19, wherein PB-mediated CRISPR system is introduced into mouse by hydrodynamic tail vein injection.

21. The method of claim 19, wherein PB-mediated CRISPR system is introduced by transfection in vivo such as nanoparticles and electroporation.


SEQUENCE LISTING (177 sequences)

SEQ ID NO: 1 - DNA, 74 bp, Artificial Sequence
  Oligonucleotide
atatcttgtggaaaggacgaaacaccgcggtgcagattcgaactgcggttttagagctagaaatagcaagttaa

SEQ ID NO: 2 - DNA, 74 bp, Artificial Sequence
  Oligonucleotide
atatcttgtggaaaggacgaaacaccgtgagggcttaccatcaccatgttttagagctagaaatagcaagttaa

SEQ ID NO: 3 - DNA, 74 bp, Artificial Sequence
  Oligonucleotide
atatcttgtggaaaggacgaaacaccggcagcacgacaagcgtgtccgttttagagctagaaatagcaagttaa

SEQ ID NO: 4 - DNA, 24 bp, Artificial Sequence
  Oligonucleotide
caccggctgctgtcagggagctca

SEQ ID NO: 5 - DNA, 24 bp, Artificial Sequence
  Oligonucleotide
aaactgagctccctgacagcagcc

SEQ ID NO: 6 - DNA, 24 bp, Artificial Sequence
  Oligonucleotide
caccgaaagtgccaacagatatcc

SEQ ID NO: 7 - DNA, 24 bp, Artificial Sequence
  Oligonucleotide
aaacggatatctgttggcactttc

SEQ ID NO: 8 - DNA, 20 bp, Artificial Sequence
  Oligonucleotide
ccgaagaggtcgtgaagaag

SEQ ID NO: 9 - DNA, 20 bp, Artificial Sequence
  Oligonucleotide
gccttatccagttcgctcag

SEQ ID NO: 10 - DNA, 20 bp, Artificial Sequence
  Oligonucleotide
acagtgccatgagagaccaa

SEQ ID NO: 11 - DNA, 21 bp, Artificial Sequence
  Oligonucleotide
ctcgcttaatctgctccctgt

SEQ ID NO: 12 - DNA, 21 bp, Artificial Sequence
  Oligonucleotide
cttcaacagcaactcccactc

SEQ ID NO: 13 - DNA, 21 bp, Artificial Sequence
  Oligonucleotide
cctgttgctgtagccgtattc

SEQ ID NO: 14 - DNA, 27 bp, Artificial Sequence
  Oligonucleotide
atatcttgtggaaaggacgaaacaccg

SEQ ID NO: 15 - DNA, 27 bp, Artificial Sequence
  Oligonucleotide
ttaacttgctatttctagctctaaaac

SEQ ID NO: 16 - DNA, 24 bp, Artificial Sequence
  Oligonucleotide
caccggctgctgtcagggagctca

SEQ ID NO: 17 - DNA, 24 bp, Artificial Sequence
  Oligonucleotide
aaactgagctccctgacagcagcc

SEQ ID NO: 18 - DNA, 24 bp, Artificial Sequence
  Oligonucleotide
caccgaaagtgccaacagatatcc

SEQ ID NO: 19 - DNA, 24 bp, Artificial Sequence
  Oligonucleotide
aaacggatatctgttggcactttc

SEQ ID NO: 20 - DNA, 22 bp, Artificial Sequence
  Oligonucleotide
gtcagaagcttttggaccaact

SEQ ID NO: 21 - DNA, 22 bp, Artificial Sequence
  Oligonucleotide
acaatcccagttcggcttaaag

SEQ ID NO: 22 - DNA, 24 bp, Artificial Sequence
  Oligonucleotide
atatcgaaagtttggcctcagcgt

SEQ ID NO: 23 - DNA, 24 bp, Artificial Sequence
  Oligonucleotide
acgggccactttcgattccgggta

SEQ ID NO: 24 - DNA, 24 bp, Artificial Sequence
  Oligonucleotide
tgtggtctcatgatggtcaagtag

SEQ ID NO: 25 - DNA, 24 bp, Artificial Sequence
  Oligonucleotide
tggctcatggtattgaatggctga

SEQ ID NO: 26 - DNA, 24 bp, Artificial Sequence
  Oligonucleotide
tcttgggtttcaccagcacgtcct

SEQ ID NO: 27 - DNA, 23 bp, Artificial Sequence
  Oligonucleotide
tccagggtagcttcaagatccaa

SEQ ID NO: 28 - DNA, 24 bp, Artificial Sequence
  Oligonucleotide
agcaacttcatggtggctcacaac

SEQ ID NO: 29 - DNA, 24 bp, Artificial Sequence
  Oligonucleotide
ttcacccccttctttctgtggagc

SEQ ID NO: 30 - DNA, 25 bp, Artificial Sequence
  Oligonucleotide
acattgtccctcacttcatcctcca

SEQ ID NO: 31 - DNA, 24 bp, Artificial Sequence
  Oligonucleotide
agcctaagtatgtgacacgccttt

SEQ ID NO: 32 - DNA, 24 bp, Artificial Sequence
  Oligonucleotide
aggaatggaggggaggaaggaaga

SEQ ID NO: 33 - DNA, 24 bp, Artificial Sequence
  Oligonucleotide
tgaggcctggtagttatgttagag

SEQ ID NO: 34 - DNA, 24 bp, Artificial Sequence
  Oligonucleotide
ttgtgtgggttgagtgccgtactg

SEQ ID NO: 35 - DNA, 24 bp, Artificial Sequence
  Oligonucleotide
atacacatgatcccaccactcagg

SEQ ID NO: 36 - DNA, 24 bp, Artificial Sequence
  Oligonucleotide
gtgagtgctgatgagattttcaag

SEQ ID NO: 37 - DNA, 24 bp, Artificial Sequence
  Oligonucleotide
tccaagtgtaactatggaatggtg

SEQ ID NO: 38 - DNA, 24 bp, Artificial Sequence
  Oligonucleotide
aagattgggaaagtcacttcggcc

SEQ ID NO: 39 - DNA, 24 bp, Artificial Sequence
  Oligonucleotide
gcaaacttcaagtcctagcaacag

SEQ ID NO: 40 - DNA, 24 bp, Artificial Sequence
  Oligonucleotide
tttgctcccctgtttcctccacat

SEQ ID NO: 41 - DNA, 24 bp, Artificial Sequence
  Oligonucleotide
acagctcaagatcaagacttgctg

SEQ ID NO: 42 - DNA, 24 bp, Artificial Sequence
  Oligonucleotide
acagtttcccctcttgcatctcgt

SEQ ID NO: 43 - DNA, 24 bp, Artificial Sequence
  Oligonucleotide
gagaccatgaaagcaaataccgag

SEQ ID NO: 44 - DNA, 24 bp, Artificial Sequence
  Oligonucleotide
tatggggtggaggagaactgtgag

SEQ ID NO: 45 - DNA, 24 bp, Artificial Sequence
  Oligonucleotide
gctcctatcaagaactcttgggca

SEQ ID NO: 46 - DNA, 24 bp, Artificial Sequence
  Oligonucleotide
tgcgcaggcgcattgatgcagttt

SEQ ID NO: 47 - DNA, 24 bp, Artificial Sequence
  Oligonucleotide
gatagacatcaggactggtgagga

SEQ ID NO: 48 - DNA, 24 bp, Artificial Sequence
  Oligonucleotide
ttcatttcttcctgatcggtctcc

SEQ ID NO: 49 - DNA, 24 bp, Artificial Sequence
  Oligonucleotide
ataacctagcattcaaggtgcaga

SEQ ID NO: 50 - DNA, 24 bp, Artificial Sequence
  Oligonucleotide
aactcaccaggagaagagtgtgag

SEQ ID NO: 51 - DNA, 25 bp, Artificial Sequence
  Oligonucleotide
tgctgtgtactatatcaggtatggc

SEQ ID NO: 52 - DNA, 24 bp, Artificial Sequence
  Oligonucleotide
aggagcgttctaggacatcatgtg

SEQ ID NO: 53 - DNA, 24 bp, Artificial Sequence
  Oligonucleotide
ctgacataagcaacctcaggaatg

SEQ ID NO: 54 - DNA, 24 bp, Artificial Sequence
  Oligonucleotide
aacactgagacagctcagttccca

SEQ ID NO: 55 - DNA, 24 bp, Artificial Sequence
  Oligonucleotide
tgagctccgcagtacagtctttgt

SEQ ID NO: 56 - DNA, 24 bp, Artificial Sequence
  Oligonucleotide
taggtagatgaggacagacagaca

SEQ ID NO: 57 - DNA, 24 bp, Artificial Sequence
  Oligonucleotide
tgcagcctccaagagggattgttt

SEQ ID NO: 58 - DNA, 24 bp, Artificial Sequence
  Oligonucleotide
cttaccatagtagaagggactgtc

SEQ ID NO: 59 - DNA, 24 bp, Artificial Sequence
  Oligonucleotide
atctggtaggcctaggatcagggt

SEQ ID NO: 60 - DNA, 24 bp, Artificial Sequence
  Oligonucleotide
aagagtgactccctttcttagtgc

SEQ ID NO: 61 - DNA, 24 bp, Artificial Sequence
  Oligonucleotide
tataaccttcctttctgtggtcct

SEQ ID NO: 62 - DNA, 24 bp, Artificial Sequence
  Oligonucleotide
tgcatggagattaacctgggtcaa

SEQ ID NO: 63 - DNA, 24 bp, Artificial Sequence
  Oligonucleotide
aaccagcagcaacaagaaacaccc

SEQ ID NO: 64 - DNA, 24 bp, Artificial Sequence
  Oligonucleotide
atcagctatctcaggtatctcagg

SEQ ID NO: 65 - DNA, 24 bp, Artificial Sequence
  Oligonucleotide
ttcctgattcgcagaaccagacca

SEQ ID NO: 66 - DNA, 24 bp, Artificial Sequence
  Oligonucleotide
aggggatagaacttatgtggaggt

SEQ ID NO: 67 - DNA, 24 bp, Artificial Sequence
  Oligonucleotide
tgaattggtgggatcagaagtgga

SEQ ID NO: 68 - DNA, 24 bp, Artificial Sequence
  Oligonucleotide
atggtaggcacctggaagttcaac

SEQ ID NO: 69 - DNA, 24 bp, Artificial Sequence
  Oligonucleotide
atctccctcaaccagagtgatctc

SEQ ID NO: 70 - DNA, 24 bp, Artificial Sequence
  Oligonucleotide
tccagctaccagcaacagaagaat

SEQ ID NO: 71 - DNA, 24 bp, Artificial Sequence
  Oligonucleotide
ccaaggaagagtagacatcaacct

SEQ ID NO: 72 - DNA, 24 bp, Artificial Sequence
  Oligonucleotide
acgagtgcccttcagagagcagag

SEQ ID NO: 73 - DNA, 24 bp, Artificial Sequence
  Oligonucleotide
tgacttagaggttcagcttgatgc

SEQ ID NO: 74 - DNA, 24 bp, Artificial Sequence
  Oligonucleotide
tactaaatctccttggtgatcccc

SEQ ID NO: 75 - DNA, 24 bp, Artificial Sequence
  Oligonucleotide
tttcttattgcttcacctgtggag

SEQ ID NO: 76 - DNA, 24 bp, Artificial Sequence
  Oligonucleotide
aggaccttggtgtctctttaggac

SEQ ID NO: 77 - DNA, 24 bp, Artificial Sequence
  Oligonucleotide
ccggatctttccttgttctgctaa

SEQ ID NO: 78 - DNA, 24 bp, Artificial Sequence
  Oligonucleotide
agaggcaacaagccgatgagggaa

SEQ ID NO: 79 - DNA, 24 bp, Artificial Sequence
  Oligonucleotide
tagcttgttcctgacctgcctgta

SEQ ID NO: 80 - DNA, 24 bp, Artificial Sequence
  Oligonucleotide
aggttgaaagagctttgcgtctcc

SEQ ID NO: 81 - DNA, 24 bp, Artificial Sequence
  Oligonucleotide
gatgcagttctctgttcaaccaac

SEQ ID NO: 82 - DNA, 24 bp, Artificial Sequence
  Oligonucleotide
agtaacctatatgtagtcccatcc

SEQ ID NO: 83 - DNA, 24 bp, Artificial Sequence
  Oligonucleotide
tgaccctgtgttggttttcatcct

SEQ ID NO: 84 - DNA, 24 bp, Artificial Sequence
  Oligonucleotide
aacagtccagagacccagacaatg

SEQ ID NO: 85 - DNA, 24 bp, Artificial Sequence
  Oligonucleotide
tgtacagctgataactgtgtcctg

SEQ ID NO: 86 - DNA, 24 bp, Artificial Sequence
  Oligonucleotide
attgggagacaaagtggatgctga

SEQ ID NO: 87 - DNA, 24 bp, Artificial Sequence
  Oligonucleotide
tgttcattaagactacagcagtgg

SEQ ID NO: 88 - DNA, 24 bp, Artificial Sequence
  Oligonucleotide
aagaagtcaggcaagcacttcagg

SEQ ID NO: 89 - DNA, 24 bp, Artificial Sequence
  Oligonucleotide
aacacttgagttttgccagcccca

SEQ ID NO: 90 - DNA, 24 bp, Artificial Sequence
  Oligonucleotide
tggagaaacccagagacctttatc

SEQ ID NO: 91 - DNA, 24 bp, Artificial Sequence
  Oligonucleotide
acccacaattcaagagtacagtgg

SEQ ID NO: 92 - DNA, 24 bp, Artificial Sequence
  Oligonucleotide
agaggcaacaagccgatgagggaa

SEQ ID NO: 93 - DNA, 24 bp, Artificial Sequence
  Oligonucleotide
tagcttgttcctgacctgcctgta

SEQ ID NO: 94 - DNA, 24 bp, Artificial Sequence
  Oligonucleotide
tgagggacttaaaagtctttcacc

SEQ ID NO: 95 - DNA, 23 bp, Artificial Sequence
  Oligonucleotide
cgaatggtgccaaatcggtcaaa

SEQ ID NO: 96 - DNA, 24 bp, Artificial Sequence
  Oligonucleotide
tgctgctctgggatacagagacta

SEQ ID NO: 97 - DNA, 24 bp, Artificial Sequence
  Oligonucleotide
tttccaaaccaggttgcagtttgg

SEQ ID NO: 98 - DNA, 24 bp, Artificial Sequence
  Oligonucleotide
aaggccagcctgagctacacaaag

SEQ ID NO: 99 - DNA, 24 bp, Artificial Sequence
  Oligonucleotide
ataagcctctggctgctaaggcct

SEQ ID NO: 100 - DNA, 24 bp, Artificial Sequence
  Oligonucleotide
tggggaacaaggtttacatagcat

SEQ ID NO: 101 - DNA, 24 bp, Artificial Sequence
  Oligonucleotide
acagatcacttcaagtgtcctgct

SEQ ID NO: 102 - DNA, 24 bp, Artificial Sequence
  Oligonucleotide
catgttccacatggcatgtgtatc

SEQ ID NO: 103 - DNA, 24 bp, Artificial Sequence
  Oligonucleotide
aaatctccagccaataggaacgga

SEQ ID NO: 104 - DNA, 24 bp, Artificial Sequence
  Oligonucleotide
tgcactggtgtacgaagtcaccct

SEQ ID NO: 105 - DNA, 24 bp, Artificial Sequence
  Oligonucleotide
attacctctgagctgtaccactca

SEQ ID NO: 106 - DNA, 24 bp, Artificial Sequence
  Oligonucleotide
tgaagtgtcatcagaggtagctct

SEQ ID NO: 107 - DNA, 24 bp, Artificial Sequence
  Oligonucleotide
aactcactttgtagaccaggctgg

SEQ ID NO: 108 - DNA, 24 bp, Artificial Sequence
  Oligonucleotide
agtgttggcttctttctatgactg

SEQ ID NO: 109 - DNA, 24 bp, Artificial Sequence
  Oligonucleotide
tgcagaacgctgcagctcagtgcc

SEQ ID NO: 110 - DNA, 24 bp, Artificial Sequence
  Oligonucleotide
agccaaagatccgtaccacttggc

SEQ ID NO: 111 - DNA, 24 bp, Artificial Sequence
  Oligonucleotide
ttctgaatgaatgtgtgagggtac

SEQ ID NO: 112 - DNA, 24 bp, Artificial Sequence
  Oligonucleotide
acagacagacaaacatacatacag

SEQ ID NO: 113 - DNA, 24 bp, Artificial Sequence
  Oligonucleotide
acctgaggtctccactagcctgat

SEQ ID NO: 114 - DNA, 25 bp, Artificial Sequence
  Oligonucleotide
tgttccaggtcaccagtctaggaag

SEQ ID NO: 115 - DNA, 24 bp, Artificial Sequence
  Oligonucleotide
atgccactcatccaaaagtcaacc

SEQ ID NO: 116 - DNA, 24 bp, Artificial Sequence
  Oligonucleotide
ttttggccaggtgaattgataggc

SEQ ID NO: 117 - DNA, 24 bp, Artificial Sequence
  Oligonucleotide
aataagacctcggtacaaacctgc

SEQ ID NO: 118 - DNA, 24 bp, Artificial Sequence
  Oligonucleotide
ttcaaaaactccgatgacccgatc

SEQ ID NO: 119 - DNA, 24 bp, Artificial Sequence
  Oligonucleotide
gtccactttaggactatgaacagc

SEQ ID NO: 120 - DNA, 24 bp, Artificial Sequence
  Oligonucleotide
tttaccccctccgtgttatgtgtc

SEQ ID NO: 121 - DNA, 24 bp, Artificial Sequence
  Oligonucleotide
atgtctgacactgaagcagaactg

SEQ ID NO: 122 - DNA, 24 bp, Artificial Sequence
  Oligonucleotide
agcttgccaattgaacagtgtatg

SEQ ID NO: 123 - DNA, 24 bp, Artificial Sequence
  Oligonucleotide
tactctccttccttgccagataag

SEQ ID NO: 124 - DNA, 24 bp, Artificial Sequence
  Oligonucleotide
tctacccacctgtacccagtaatg

SEQ ID NO: 125 - DNA, 24 bp, Artificial Sequence
  Oligonucleotide
catgagaactattgggttgtgtgg

SEQ ID NO: 126 - DNA, 23 bp, Artificial Sequence
  Oligonucleotide
aactgcatcccagcagggtacat

SEQ ID NO: 127 - DNA, 24 bp, Artificial Sequence
  Oligonucleotide
tccacttaagcttcggaagacccc

SEQ ID NO: 128 - DNA, 24 bp, Artificial Sequence
  Oligonucleotide
aagtgcttcagctttgggagtgct

SEQ ID NO: 129 - DNA, 23 bp, Artificial Sequence
  Oligonucleotide
atgtactgaggacacaggtggag

SEQ ID NO: 130 - DNA, 23 bp, Artificial Sequence
  Oligonucleotide
gttgatattctggatcaatgtcc

SEQ ID NO: 131 - DNA, 24 bp, Artificial Sequence
  Oligonucleotide
agagttttgggctgcaagtccagc

SEQ ID NO: 132 - DNA, 24 bp, Artificial Sequence
  Oligonucleotide
tagctatagaagttgtttgccatg

SEQ ID NO: 133 - DNA, 24 bp, Artificial Sequence
  Oligonucleotide
tgtactctgcagacacctgctttc

SEQ ID NO: 134 - DNA, 24 bp, Artificial Sequence
  Oligonucleotide
gtacttctcattgtggccttgatc

SEQ ID NO: 135 - DNA, 24 bp, Artificial Sequence
  Oligonucleotide
gatgaacaagatcagcatctatac

SEQ ID NO: 136 - DNA, 23 bp, Artificial Sequence
  Oligonucleotide
cacagtgtcaccacaatgtttcc

SEQ ID NO: 137 - DNA, 24 bp, Artificial Sequence
  Oligonucleotide
acgaggcagtttgctttaggaagg

SEQ ID NO: 138 - DNA, 24 bp, Artificial Sequence
  Oligonucleotide
tgtcacgcaatgattccactctga

SEQ ID NO: 139 - DNA, 24 bp, Artificial Sequence
  Oligonucleotide
tgacctctggtccatctctttcat

SEQ ID NO: 140 - DNA, 24 bp, Artificial Sequence
  Oligonucleotide
agcactacctgcactgaccggaaa

SEQ ID NO: 141 - DNA, 24 bp, Artificial Sequence
  Oligonucleotide
agacttcagaagcatggagagcac

SEQ ID NO: 142 - DNA, 24 bp, Artificial Sequence
  Oligonucleotide
ctgcaaaacagagtcctagctctg

SEQ ID NO: 143 - DNA, 24 bp, Artificial Sequence
  Oligonucleotide
tggcccaagccactcttctagatt

SEQ ID NO: 144 - DNA, 24 bp, Artificial Sequence
  Oligonucleotide
tatttccgggatcacatgtccttg

SEQ ID NO: 145 - DNA, 24 bp, Artificial Sequence
  Oligonucleotide
tgtccccttggtttctatacctac

SEQ ID NO: 146 - DNA, 24 bp, Artificial Sequence
  Oligonucleotide
aatttgctaggcaggcagcctatg

SEQ ID NO: 147 - DNA, 42 bp, Artificial Sequence
  Oligonucleotide
gtttctatcagagcgaggcgtgaaagtatttcgatttcttgg

SEQ ID NO: 148 - DNA, 43 bp, Artificial Sequence
  Oligonucleotide
gtttctatcagagcgaggcggttgataacggactagccttatt

SEQ ID NO: 149 - DNA, 42 bp, Artificial Sequence
  Oligonucleotide
gtttctatcactatggtggctgaaagtatttcgatttcttgg

SEQ ID NO: 150 - DNA, 43 bp, Artificial Sequence
  Oligonucleotide
gtttctatcactatggtggcgttgataacggactagccttatt

SEQ ID NO: 151 - DNA, 42 bp, Artificial Sequence
  Oligonucleotide
gtttctatcaatgccagttttgaaagtatttcgatttcttgg

SEQ ID NO: 152 - DNA, 43 bp, Artificial Sequence
  Oligonucleotide
gtttctatcaatgccagtttgttgataacggactagccttatt

SEQ ID NO: 153 - DNA, 42 bp, Artificial Sequence
  Oligonucleotide
gtttctatcagcgcccgacatgaaagtatttcgatttcttgg

SEQ ID NO: 154 - DNA, 43 bp, Artificial Sequence
  Oligonucleotide
gtttctatcagcgcccgacagttgataacggactagccttatt

SEQ ID NO: 155 - DNA, 42 bp, Artificial Sequence
  Oligonucleotide
gtttctatcatgatccgtagtgaaagtatttcgatttcttgg

SEQ ID NO: 156 - DNA, 43 bp, Artificial Sequence
  Oligonucleotide
gtttctatcatgatccgtaggttgataacggactagccttatt

SEQ ID NO: 157 - DNA, 42 bp, Artificial Sequence
  Oligonucleotide
gtttctatcaaaggtgcccttgaaagtatttcgatttcttgg

SEQ ID NO: 158 - DNA, 43 bp, Artificial Sequence
  Oligonucleotide
gtttctatcaaaggtgccctgttgataacggactagccttatt

SEQ ID NO: 159 - DNA, 42 bp, Artificial Sequence
  Oligonucleotide
gtttctatcaggggttgcattgaaagtatttcgatttcttgg

SEQ ID NO: 160 - DNA, 43 bp, Artificial Sequence
  Oligonucleotide
gtttctatcaggggttgcatgttgataacggactagccttatt

SEQ ID NO: 161 - DNA, 42 bp, Artificial Sequence
  Oligonucleotide
gtttctatcatttgaccgcgtgaaagtatttcgatttcttgg

SEQ ID NO: 162 - DNA, 43 bp, Artificial Sequence
  Oligonucleotide
gtttctatcatttgaccgcggttgataacggactagccttatt

SEQ ID NO: 163 - DNA, 42 bp, Artificial Sequence
  Oligonucleotide
gtttctatcacgtgagtctatgaaagtatttcgatttcttgg

SEQ ID NO: 164 - DNA, 43 bp, Artificial Sequence
  Oligonucleotide
gtttctatcacgtgagtctagttgataacggactagccttatt

SEQ ID NO: 165 - DNA, 42 bp, Artificial Sequence
  Oligonucleotide
gtttctatcagggtgaaagctgaaagtatttcgatttcttgg

SEQ ID NO: 166 - DNA, 43 bp, Artificial Sequence
  Oligonucleotide
gtttctatcagggtgaaagcgttgataacggactagccttatt

SEQ ID NO: 167 - DNA, 24 bp, Artificial Sequence
  Oligonucleotide
attctgccagctggcgaagacgtg

SEQ ID NO: 168 - DNA, 24 bp, Artificial Sequence
  Oligonucleotide
actcgggatacaaatttccttcca

SEQ ID NO: 169 - DNA, 24 bp, Artificial Sequence
  Oligonucleotide
tgagtcttcttagtttagggatgg

SEQ ID NO: 170 - DNA, 24 bp, Artificial Sequence
  Oligonucleotide
cagttgctgcctcctagtgtacct

SEQ ID NO: 171 - DNA, 24 bp, Artificial Sequence
  Oligonucleotide
agctgacaccgggagtcctgatgg

SEQ ID NO: 172 - DNA, 24 bp, Artificial Sequence
  Oligonucleotide
ttgtaggatcaaaggctgaccctg

SEQ ID NO: 173 - DNA, 24 bp, Artificial Sequence
  Oligonucleotide
aaggaaagtagtgctagcctttgc

SEQ ID NO: 174 - DNA, 24 bp, Artificial Sequence
  Oligonucleotide
aatcactctacctccctggctcta

SEQ ID NO: 175 - DNA, 24 bp, Artificial Sequence
  Oligonucleotide
accaatgctgtccagtccatttgc

SEQ ID NO: 176 - DNA, 24 bp, Artificial Sequence
  Oligonucleotide
taggcaagcagacaacactaatgt

SEQ ID NO: 177 - DNA, 24 bp, Artificial Sequence
  Oligonucleotide
tcttctcaggctgagagtcaatcc